(19) United States
(12) Patent Application Publication
(10) Pub. No.: US 2025/0345194 A1
(43) Pub. Date: Nov. 13, 2025

(54) FLOW DIVERTING STENT DELIVERY SYSTEM INCLUDING A TORQUEABLE WIRE

(71) Applicant: Deepin Technologies, LLC, San Jose, CA (US)

(72) Inventor: Jon Schabert, San Jose, CA (US)

(73) Assignee: Deepin Technologies, LLC, San Jose, CA (US)

(21) Appl. No.: 19/272,820

(22) Filed: Jul. 17, 2025

Related U.S. Application Data
(63) Continuation-in-part of application No. 18/754,487, filed on Jun. 26, 2024.
(60) Provisional application No. 63/581,536, filed on Sep. 8, 2023.
(60) Provisional application No. 63/675,596, filed on Jul. 25, 2024.
(60) Provisional application No. 63/675,592, filed on Jul. 25, 2024.

(51) Int. Cl. A61F 2/95

(52) U.S. Cl. CPC A61F 2/95

(57) ABSTRACT

An endovascular system includes a delivery device operable to deliver a tubular implant through a catheter. The delivery device comprises a delivery wire, a set of stoppers comprising a distal stopper and a proximal stopper fixedly attached to the delivery wire, a coupler disposed between the distal stopper and the proximal stopper, and a protection cover wrapping at least an end portion of the tubular implant. A method of delivering a tubular implant is also described.

### CROSS-REFERENCE TO RELATED APPLICATIONS

This application is a continuation-in-part of U.S. Ser. No. 18/754,487 filed Jun. 26, 2024 entitled “Delivery System for Tubular Braided Devices,” which claims priority to U.S. provisional patent application No. 63/581,536 filed Sep. 8, 2023 entitled “Delivery System for Tubular Braided Devices,” the disclosures of both of which are hereby incorporated by reference in their entirety.

This application claims priority to U.S. provisional patent application No. 63/675,596 filed Jul. 25, 2024 entitled “Flow Diverting Stent Delivery System including a Torqueable Wire” and U.S. provisional patent application No. 63/675,592 filed Jul. 25, 2024 entitled “Braided Devices Including MP35N-Nitinol DFT,” the disclosures of both of which are hereby incorporated by reference in their entirety.

### TECHNICAL FIELD

This application relates generally to medical devices and methods of using medical devices to treat diseases. In particular, various embodiments of an endovascular system, a delivery device, and a method for delivering an implant to a treatment site such as in a cerebral vasculature of a patient are described.

### BACKGROUND

Stents or flow diverting stents are commonly used in endovascular interventions to treat vascular diseases. Stents can be constructed from patterned cut tubes or braided wires or filaments. In cerebral vascular interventions, self-expanding braided stents are typically used for their desirable resheathability, conformability, and radial force. Braided stents generally comprise a tubular or cylindrical structure constructed from a plurality of wires or filaments interlaced or woven in a repeated pattern.

Regardless of stent construction, a stent to be implanted in a cerebral vessel need be delivered in a collapsed state through the neuro vasculature and then allowed to be expanded to the target vessel size. Delivery of a stent is typically achieved by having the stent compressed or constrained in a sheath or catheter and mounted over a delivery system to facilitate movement of the stent.

In conventional delivery approaches, a delivery system is coupled to a compressed stent at the proximal end of the stent. Advancement of the stent distally requires pushing on the stent whereas retraction of the stent proximally requires pulling on the stent, both from the proximal end of the stent. In some conventional delivery systems, bumpers are provided at the proximal end and the distal end of a stent. The stent is advanced by pushing the bumper at the proximal end and retracted by pushing the bumper against the distal end respectively.

Stent delivery using conventional approaches can often be difficult due to the tortuosity of the cerebral vessel anatomy and the need for stents having a larger expanded diameter or higher radial force. Conventional systems often require excessive force and effort to deliver stents and experience various other issues.

Therefore, while advancement has been made in vascular interventions, there is still a general need for improvement of delivery systems and methods to overcome these and other issues experienced by the conventional approaches.

### SUMMARY

In one aspect, embodiments of the disclosure feature an endovascular system. In general, an embodiment of the endovascular system comprises a catheter, a tubular implant, and a delivery device. The tubular implant has a collapsed state for being disposed in the lumen of the catheter and an expanded state when unconstrained by the catheter. The delivery device is operable to advance and/or retract the tubular implant relative to the catheter. The delivery device comprises a delivery wire, a first set of stoppers comprising a distal stopper and a proximal stopper fixedly attached to the delivery wire, a first coupler disposed between the distal stopper and the proximal stopper of the first set, a second set of stoppers comprising a distal stopper and a proximal stopper fixedly attached to the delivery wire, and a second coupler disposed between the distal stopper and the proximal stopper of the second set. The first coupler is configured to contact and apply an outwardly radial force to a distal end portion of the tubular implant in the lumen of the catheter to grip the tubular implant in the lumen of the catheter, and the second coupler is configured to contact and apply an outwardly radial force to a proximal end portion of the tubular implant in the lumen of the catheter to grip the tubular implant in the lumen of the catheter. The proximal stopper of the first set is configured to engage the first coupler when the delivery wire is advanced to apply a translating force in a distal direction to the first coupler thereby generating a pulling force in the distal direction on a portion of the tubular implant proximal of the distal end portion of the tubular implant. The distal stopper of the second set is configured to engage the second coupler when the delivery wire is retracted to apply a translating force in a proximal direction to the second coupler thereby generating a pulling force in the proximal direction on a portion of the tubular implant distal of the proximal end portion of the tubular implant.

In another aspect, embodiments of the disclosure feature an endovascular system. In general, an embodiment of the endovascular system comprises a catheter, a tubular implant, and a delivery device. The tubular implant has a collapsed state for being disposed in the lumen of the catheter and an expanded state when unconstrained by the catheter. The delivery device is operable to advance and/or retract the tubular implant relative to the catheter. The delivery device comprises a delivery wire, a set of stoppers comprising a distal stopper and a proximal stopper fixedly attached to the delivery wire, and a coupler disposed between the distal stopper and the proximal stopper of the set. The coupler is configured to contact and apply an outwardly radial force to a distal end portion of the tubular implant in the lumen of the catheter to grip the tubular implant in the lumen of the catheter. The proximal stopper of the set is configured to engage the coupler when the delivery wire is advanced to apply a translating force in a distal direction to the coupler thereby generating a pulling force in the distal direction on a portion of the tubular implant proximal of the distal end portion of the tubular implant.

In a further aspect, embodiments of the disclosure feature a method of delivering a tubular implant in a lumen of a catheter. In general, an embodiment of the method comprises the following steps: applying a translating force in a distal direction to a distal end portion of the tubular implant thereby generating a pulling force on a portion of the tubular implant proximal of the distal end portion of the tubular implant, and applying a translating force in the distal direction to a proximal end portion of the tubular implant.

In another aspect, embodiments of the disclosure feature an endovascular system. In general, an embodiment of the endovascular system comprises a catheter having a lumen, a tubular implant having a collapsed state for being disposed in the lumen of the catheter and an expanded state when unconstrained by the catheter, and a delivery device operable to deliver the tubular implant through the catheter. The delivery device comprises a delivery wire having a distal section and a proximal section, a set of stoppers comprising a distal stopper and a proximal stopper fixedly attached to the distal section of the delivery wire respectively, a coupler disposed between the distal stopper and the proximal stopper and configured to apply an outwardly radial force to a distal portion of the tubular implant in the lumen of the catheter to grip the tubular implant, and a protection cover comprising a first end portion fixedly coupled to the distal section of the delivery wire and a second end portion wrapping at least an end portion of the tubular implant in the lumen of the catheter. The coupler is provided with a through-opening allowing the distal section of the delivery wire to pass through and configured to allow the delivery wire to slide longitudinally relative to the coupler in the lumen of the catheter to thereby allow the proximal stopper and/or the distal stopper to engage and apply a translating force to the coupler to advance and/or withdraw the tubular implant, and to allow the delivery wire to rotate relative to the coupler to thereby remove the protection cover off the tubular implant.

In a further aspect, embodiments of the disclosure feature a method of delivering a tubular implant. In general, an embodiment of the method comprises a step of positioning an endovascular system in a vessel of a patient. The endovascular system comprises a catheter having a lumen, a tubular implant constrained in the lumen of the catheter, and a delivery device. The delivery device comprises a delivery wire, a set of stoppers comprising a distal stopper and a proximal stopper fixedly attached to the delivery wire, a coupler between the distal stopper and the proximal stopper configured to contact and apply an outwardly radial force to a distal portion of the tubular implant in the lumen of the catheter to grip the tubular implant, and a protection cover comprising a first end portion fixedly coupled to the delivery wire and a second end portion wrapping at least an end portion of the tubular implant in the lumen of the catheter. The method further comprises the following steps: advancing or unsheathing the tubular implant by relative movement of the catheter and the deliver wire to allow the proximal stopper to engage and apply a translating force to the coupler, whereby the end portion of the tubular implant wrapped by the second end portion of the protection cover exits the lumen of the catheter, and rotating the delivery wire to allow the protection cover to twist, whereby the second end portion of the protection cover is removed off from the end portion of the tubular implant.

This Summary is provided to introduce selected aspects and embodiments of this disclosure in a simplified form and is not intended to identify key features or essential characteristics of the claimed subject matter, nor is it intended to be used as an aid in determining the scope of the claimed subject matter. The selected aspects and embodiments are presented merely to provide the reader with a summary of certain forms the invention might take and are not intended to limit the scope of the invention. Other aspects and embodiments of the disclosure are described in the section of Detailed Description.

These and various other aspects, embodiments, features, and advantages of the disclosure will become better understood upon reading of the following detailed description in conjunction with the accompanying drawings.

### BRIEF DESCRIPTION OF THE DRAWINGS

FIG. 1 is a simplified illustration of an example endovascular system according to embodiments of the disclosure.

FIG. 2 is a simplified illustration of an example delivery device according to embodiments of the disclosure.

FIG. 3 is a schematic illustrating the use of an example delivery device to advance a tubular implant according to embodiments of the disclosure.

FIG. 4 is a schematic illustrating the use of an example delivery device to retract a tubular implant according to embodiments of the disclosure.

FIG. 5 is a simplified illustration of an example delivery device according to alternative embodiments of the disclosure.

FIG. 6 depicts an example application of an endovascular system of the disclosure in treating an aneurysm in a cerebral vasculature according to embodiments of the disclosure.

FIG. 7 is a flowchart illustrating an example method according to embodiments of the disclosure.

FIG. 8 is a simplified illustration of an example endovascular system according to alternative embodiments of the disclosure.

FIG. 9 is a simplified illustration of an example delivery device according to alternative embodiments of the disclosure.

FIG. 10 is a flowchart illustrating an example method according to embodiments of the disclosure.

FIG. 11 is a schematic illustrating an example method of delivering a tubular implant according to embodiments of the disclosure.

FIG. 12 is a schematic illustrating an example method of delivering a tubular implant according to embodiments of the disclosure.

### DETAILED DESCRIPTION OF EMBODIMENTS

With reference to the figures, various embodiments of an endovascular system, delivery device, and method will now be described. The figures are intended to facilitate description of embodiments of the disclosure and are not necessarily drawn to scale. Certain specific details may be set forth in the figures to provide a thorough understanding of the disclosure. It will be apparent to one of ordinary skill in the art that some of these specific details may not be employed to practice embodiments of the disclosure. In other instances, structures, components, systems, materials, and/or operations often associated with known medical procedures may not be shown or described in detail to avoid unnecessarily obscuring description of embodiments of the disclosure.

Embodiments of the disclosure provides an endovascular system comprising a delivery device operable to advance and/or retract a tubular implant in a catheter with reduced resistance. The delivery device allows the tubular implant to be pulled in both advancement and retraction of the implant. The pulling force can induce slight diametrical contraction of the implant, thereby reducing the overall static friction between the implant and the catheter. For example, in one embodiment of the disclosure the delivery device utilizes a distal coupling feature and a proximal coupling feature each contacting or gripping an end portion of a luminal stent constrained in a catheter. The distal coupling feature allows a pulling force in a distal direction to be generated thereby aiding advancement of the stent through the catheter. The proximal coupling feature allows a pulling force in a proximal direction to be generated thereby aiding retraction or re-sheathing of the stent. The inventive features of the disclosure ensure some tension to be applied on the stent in both advancement and retraction of the stent.

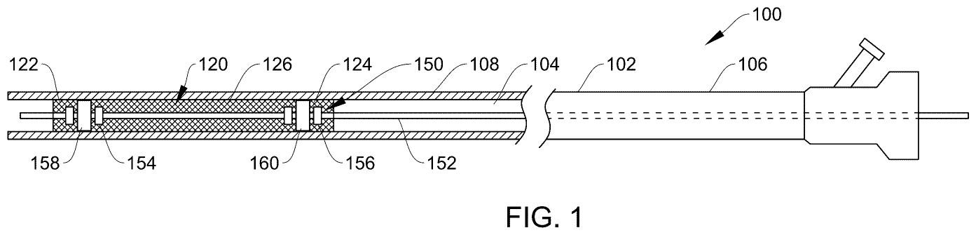

FIG. 1 depicts an endovascular system 100 according to embodiments of the disclosure. In a broad overview, the example endovascular system 100 comprises an elongate tubular member 102 having a lumen 104, a tubular implant 120 disposed in the lumen 104 of the tubular member 102, and a delivery device 150 operable to deliver and/or deploy the tubular implant 120 at a target site in a patient. The delivery device 150 generally includes an elongate delivery wire 152, one or more sets of stoppers 154, 156, and one or more coupling features 158, 160 disposed between the one or more sets of stoppers 154, 156 respectively. The one or more coupling features 158, 160 are configured to contact and apply an outwardly radial force to a portion of the implant 120 to grip the implant 120 in the lumen 104 of the tubular member 102. As will be described in greater detail below, the arrangement of the one or more coupling features 158, 160 with the one or more sets of the stoppers 154, 156 can generate a pulling force on the tubular implant 120 in the distal direction to aid advancement of the tubular implant 120, and/or, a pulling force in the proximal direction to aid retraction of the tubular implant 120.

With reference to FIG. 1, the elongate tubular member 102 can be in the form of a sheath, catheter, microcatheter, or any other suitable tubular forms. For ease of description, the term “catheter” may be used herein interchangeably with the phrase “tubular member.” The catheter 102 includes a proximal end portion 106 which may remain outside of the patient and is accessible to the user or physician when the endovascular system 100 is in use. The distal end portion 108 of the catheter 102 may be sized and dimensioned to reach a remote location in the vasculature of the patient, such as in a cerebral vessel adjacent to an aneurysm, a bifurcated blood vessel, an occlusion in a blood vessel, or the like. The lumen 104 of the catheter 102 can be sized to accommodate the tubular implant 120 and the delivery device 150 including the stoppers 154, 156 and the coupling features 158, 160. While not explicitly shown, the catheter 102 may include one or more sections or regions each of which may have different configurations and/or characteristics. For example, the distal end portion 108 of the catheter 102 may include a flexible section or region comprising a coil to provide proper bending or deflection. A flexible distal end portion 108 would allow the endovascular system 100 to navigate more easily through tortuous regions of the vasculature to remote locations in the patient. The proximal end portion 106 may be constructed from a stiffer material e.g., a rigid metal hypotube, to provide structural stability and sufficient pushability. In general, the catheter 102 or a section of the catheter 102 can be constructed from suitable biocompatible polymers, metals, or combinations thereof. The distal end portion 108 of the catheter 102 can have an outer diameter less than the outer diameter of the proximal end portion 106 to reduce the profile of the distal end portion 108 and facilitate navigation through tortuous vasculature. While not shown, the catheter 102 may include one or more markers which can be viewed e.g., via fluoroscopy, to assist the physician in operation of the endovascular system 100. The inner and outer diameters of the catheter 102 at the distal end portion 108 can be properly chosen based on applications. By way of example, for application of treating cerebral aneurysms, the catheter or microcatheter 102 may have an inner diameter ranging from 0.0165 inches to 0.040 inches for delivering a flow diverting stent of a suitable size to a target site in the cerebral anatomies or distal cerebral vessels.

With reference to FIG. 1, the tubular implant 120 can be any suitable implant compatible with the delivery device 150 of the disclosure. By way of example, the tubular implant 120 can be an embolic device such as a stent, flow diverting stent, or an intrasaccular device for treatment of brain aneurysms. The tubular implant 120 may also be a flow restoration device or a thrombectomy device etc. for treating disorders at vasculatures or other target sites in a human body. The tubular implant 120 can be expandable, having a collapsed state when compressed or constrained in the lumen 104 of the catheter 102, and an expanded state when unconstrained or deployed at a treatment site. As shown, the tubular implant 120 comprises a distal end portion 122 and a proximal end portion 124.

The tubular implant 120 can comprise a braided structure or a patterned cut structure. The braided or patterned cut structure can be a closed-cell design, in which the repeated ring structure is connected at all strut junctions. The braided or patterned cut structure can also be an open-cell stent design, in which some junctions between the repeated ring structure are removed.

An example tubular implant 120 comprises a braided stent constructed from a plurality of wires or filaments 126. The plurality of filaments 126 can be braided, woven, or interlaced in a suitable pattern. By way of example, a plurality of filaments 126 can extend spirally or helically clockwise and a plurality of filaments extend spirally or helically counterclockwise, forming a plurality of cross sections and defining a plurality of cells of a radially expandable body. The braided stent 120 can be a closed-cell design. The filaments 126 constructing the braided stent 120 can be metallic or polymeric. The filaments 126 can be radiopaque or non-radiopaque, or at least one or more of the filaments 126 making up the stent 120 are radiopaque. Depending on application, a braided stent 120 may comprise about 40 to about 96 filaments. The filaments 126 may have a diameter ranging from 0.0008 to 0.0030 inches. The filaments 126 can have a shape-memory property and/or can be heat set to form a self-expanding stent 120. In an expanded state, the braided stent 120 may have a maximal diameter ranging from 1.0 mm to 10 mm depending on applications. For treatment of cerebral aneurysms, a braided stent 120 can be constructed as a flow diverting device having a pore size and/or density suitable to disrupt or divert blood flow near an aneurysm neck, resulting in occlusion of the aneurysm while allowing blood flow in the parent and branch vessels.

Due to the interlacing helical structure, the filaments 126 making up a stent 120 can adjust position and/or orientation when the stent 120 is subjected to an external force. For example, when tension is applied to a braided stent 120 along its longitudinal axis, the helical filaments 126 reorient to align to the direction of the tensile force, resulting in reduction of the stent diameter as the stent 120 elongates or stretches. When compression is applied to the braided stent 120 along its longitudinal axis, the helical filaments 126 reorient to align perpendicularly to the direction of the compressive load, resulting in increase of the stent diameter as the stent 120 shrinks in length.

To deliver a braided stent to a target site, one common approach is to couple the proximal end of the stent to a delivery device. According to this approach, advancement of the stent distally requires pushing on the stent whereas retraction of the stent proximally requires pulling on the stent, both from the proximal end of the stent. Another approach is to utilize bumpers positioned at the stent proximal end and the stent distal end respectively. The stent constrained in a catheter is advanced by being pushed via contact with the bumper located at the proximal end, or retracted by being pushed via contact with the bumper located at the distal end. In conventional approaches, a pushing force is utilized to either advance a stent or retract the stent. However, when the stent is advanced and/or retracted, the pushing force on the stent and the resistive friction force along the stent will generate an overall compressive force on the stent. This compressive force raises the normal force between the expanding stent and the catheter constraining the stent, resulting in increased static frictional force along the stent until a sufficient pushing force is applied to overcome the frictional force to move the stent forward or rearward. Essentially, to advance and/or retract a stent by pushing involves generation of increased friction and requires application of more pushing force before the stent can move.

Embodiments of the disclosure take advantage of the properties of a braided stent or closed cell stent to reduce the amount of force needed to move the stent during delivery. The use of a distal coupling feature, or a combination of a distal coupling feature and a proximal coupling feature, allows a braided stent constrained in a catheter to be pulled in the direction of intended movement, causing the stent to slightly contract in diameter thereby reducing the overall static friction as compared to conventional push-based delivery systems.

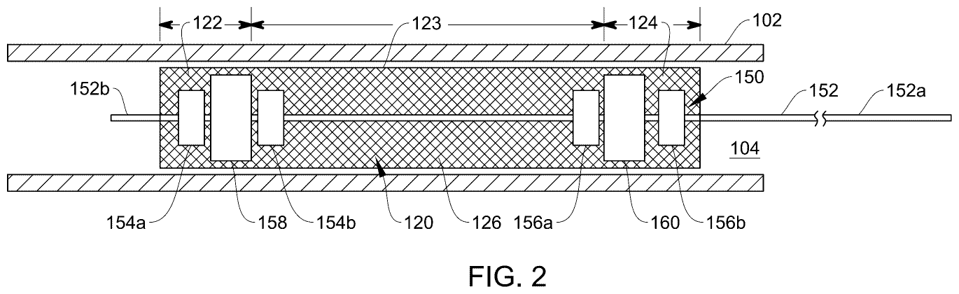

FIG. 2 depicts an example delivery device 150 according to embodiments of the disclosure. As shown, the example delivery device 150 comprises an elongate delivery wire 152, a first set of stoppers 154a, 154b affixed to the delivery wire 152, a first coupler 158 disposed between the first set of stoppers 154a, 154b, a second set of stoppers 156a, 156b affixed to the delivery wire 152, and a second coupler 160 disposed between the second set of stoppers 156a, 156b. The first coupler 158 is located adjacent to the distal end portion 122 of the tubular implant 120, and configured to contact and apply an outwardly radial force to the tubular implant 120 to grip the implant 120 in the lumen 104 of the catheter 102. The second coupler 160 is located adjacent to the proximal end portion 124 of the tubular implant 120, and configured to contact and apply an outwardly radial force to the tubular implant 120 to grip the implant 120 in the lumen 104 of the catheter 102. As will be described in greater detail below, the arrangement of the first coupler 158 and the first set stoppers 154a, 154b allows the first coupler 158, which grips the distal end portion 122 of the tubular implant 120, to generate a pulling force in the distal direction on the portion 123 of the implant 120 proximal of the distal end portion 122 of the implant 120, thus aiding advancement of the implant 120 out of the catheter 102. The arrangement of the second coupler 160 and the second set of stoppers 156a, 156b allows the second coupler 160, which grips the proximal end portion 124 of the tubular implant 120, to generate a pulling force in the proximal direction on the portion 123 of the implant 120 distal of the proximal end portion 124 of the implant 120, thus aiding retraction of the implant 120 back into the catheter 102.

With reference to FIG. 2, the elongate delivery wire 152 has a proximal end portion 152a, a distal end portion 152b, and a length extending between the proximal end portion 152a and the distal end portion 152b. The proximal end portion 152a of the delivery wire 152 may remain outside of the patient and is accessible to the physician when the delivery device 150 is in use. The proximal end portion 152a, which may be coupled to a handle, can be controlled by the user in operation. The distal end portion 152b of the delivery wire 152 can be coupled with the first set of stoppers 154a, 154b, the second set of stoppers 156a, 156b, and loaded with the tubular implant 120 to be delivered. While not specifically shown, the distal end portion 152b of the delivery wire 152 may include a section or region constructed e.g., of a coil to provide proper bending or deflection. One or more markers may also be coupled to the delivery wire 152 to aid the physician to operate the delivery device 150 via fluoroscopy. The delivery wire 152 may be constructed from a suitable metal such as stainless steel, nickel, titanium, nitinol, an alloy of metals, a biocompatible polymer, a shape memory polymer, hypotube, or any combinations thereof.

With reference to FIG. 2, the first set of stoppers 154a, 154b may comprise a stopper 154a distal of the first coupler 158 and a stopper 154b proximal of the first coupler 158. The distal stopper 154a and the proximal stopper 154b of the first set can be fixedly attached to the distal end portion 152b of the delivery wire 152 and thus can be advanced, retracted, and/or rotated with the delivery wire 152. The stoppers 154a, 154b of the first set can be sized such that they do not directly contact the tubular implant 120 in the lumen 104 of the catheter 102, or do not create, via direct contact with the tubular implant 120, a frictional force sufficient to advance or retract the implant 120 constrained in the lumen 104 of the catheter 102. In some embodiments, the stoppers 154a, 154b of the first set are sized such that their cross-section is smaller than the cross section of the first coupler 158.

The proximal stopper 154b of the first set can be configured to engage the first coupler 158 when the delivery wire 152 is advanced to apply a pushing force to the first coupler 158 from the proximal side of the first coupler 158. By way of example, the proximal stopper 154b of the first set may comprise a planar distal surface configured to engage a planar proximal surface of the first coupler 158. Other configurations and shapes of the engagement surfaces between the proximal stopper 154b of the first set and the first coupler 158 are possible and will be appreciated by one of ordinary skill in the art. These and other configurations and shapes of the engagement surfaces can be planar or curved, two-dimensional, or three-dimensional, and the scope of the disclosure is not limited to any specific configurations or shapes of the engagement surfaces between the proximal stopper of the first set and the first coupler. When the delivery wire 152 is advanced, the proximal stopper 154b of the first set engages the first coupler 158 and applies a pushing force to the first coupler 158 in the distal direction. The force applied by the proximal stopper 154b of the first set can be transferred to the distal end portion 122 of the tubular implant 120 gripped by the first coupler 158, thereby generating a pulling force on the portion 123 of the tubular implant 120 proximal of the distal end portion 122 of the implant 120 e.g., the portion 123 of the implant 120 between the first coupler 158 and the second coupler 160. Additionally, or optionally, the distal stopper 154a of the first set can be configured to engage the first coupler 158 when the delivery wire 152 is retracted. The distal stopper 154a of the first set may comprise a proximal surface, either planar or curved, or a configuration and shape, either two-dimensional or three-dimensional, for engaging the first coupler 158. In retracting or re-sheathing the tubular implant 120, the distal stopper 154a of the first set may engage the first coupler 158 and apply a pushing force to the first coupler 158 in the proximal direction from the distal side of the first coupler 158, to be described in greater detail below.

With reference to FIG. 2, the second set of stoppers 156a, 156b may comprise a stopper 156a distal of the second coupler 160 and a stopper 156b proximal of the second coupler 160. The distal stopper 156a and the proximal stopper 156b of the second set can be fixedly attached to the delivery wire 152 and thus can be advanced, retracted, and/or rotated with the delivery wire 152. The stoppers 156a, 156b of the second set can be sized such that they do not directly contact the tubular implant 120 in the lumen 104 of the catheter 102, or do not create, via direct contact with the tubular implant 120, a frictional force sufficient to retract or advance the implant 120 constrained in the lumen 104 of the catheter 102. In some embodiments, the stoppers 156a, 156b of the second set are sized such that their cross-section is smaller than the cross section of the second coupler 160.

The distal stopper 156a of the second set can be configured to engage the second coupler 160 when the delivery wire 152 is retracted to apply a pushing force to the second coupler 160 from the distal side of the second coupler 160. By way of example, the distal stopper 156a of the second set may comprise a planar proximal surface configured to engage a planar distal surface of the second coupler 160. Other configurations and shapes of the engagement surfaces between the distal stopper of the second set and the second coupler are possible and will be appreciated by one of ordinary skill in the art. These and other configurations and shapes of the engagement surfaces can be planar, curved, two-dimensional, or three-dimensional, and the scope of the disclosure is not limited to any specific configurations or shapes of the engagement surfaces between the distal stopper of the second and set the second coupler. When the delivery wire 152 is retracted, the distal stopper 156a of the second set engages the second coupler 160 and applies a pushing force to the second coupler 160 in the proximal direction. The force applied by the distal stopper 156a of the second set can be transferred to the proximal end portion 124 of the tubular implant 120 gripped by the second coupler 160, thereby generating a pulling force on the portion 123 of the tubular implant 120 distal of the proximal end portion 124 of the implant 120 e.g., the portion 123 of the implant between the first coupler 158 and the second coupler 160. Additionally, or optionally, the proximal stopper 156b of the second set can be configured to engage the second coupler 160 when the delivery wire 152 is advanced. The proximal stopper 156b of the second set may comprise a distal surface, either planar or curved, or a configuration and shape, either two-dimensional or three-dimensional, for engaging the second coupler. In advancing the tubular implant 120, the proximal stopper 156b of the second set may engage the second coupler 160 and apply a pushing force to the second coupler 160 in the distal direction from the proximal side of the second coupler 160, to be described in greater detail below.

The stoppers 154a, 154b of the first set and the stoppers 156a, 156b of the second set can be constructed of any suitable materials including polymers such as thermoplastics or thermosets, metals such as stainless steel, platinum, gold, nitinol, other alloys of metals, and any combination thereof.

With reference to FIG. 2, the first coupler 158 and the second coupler 160 are configured, e.g., sized, shaped, and/or constructed, to contact and apply an outwardly radial force to the tubular implant 120 in the collapsed state to grip the implant 120 in the lumen 104 of the tubular member 102. The first coupler 158 and the second coupler 160 can be constructed from a material that is compressible and expandable. Alternatively, the first coupler 158 or the second coupler 160 is constructed from an impressible material. By way of example, suitable materials for constructing the first coupler 158 and/or the second coupler 160 include polymeric materials e.g., elastomers such as silicone, thermosets, thermoplastics, thermoplastic urethanes, rubbers, or non-polymeric materials e.g., shape-memory metallic materials such as nitinol, stainless steel, cobalt chrome, etc.

The first coupler 158 and the second coupler 160 can be shaped and/or sized to provide a circumferential surface or surface segment conforming to the inner wall surface of the catheter 102 to allow the tubular implant 120 to be sandwiched or compressed between an inner surface of the catheter 102 and the first coupler 158 and the second coupler 160 respectively. By way of example, the first coupler 158 and/or the second coupler 160 may have a circular, semi-circular, oval, or other regular or irregular cross-sectional shape. In a specific embodiment of the disclosure, the first coupler 158 and/or the second coupler 160 are in the form of a friction pad made of an elastic polymeric material such as silicone having a circular cross-sectional shape.

According to some embodiments of the disclosure, the first coupler 158 and the second coupler 160 are configured to allow the delivery wire 152 to slide therethrough. By way of example, the first coupler 158 and the second coupler 160 may be provided with a through-passage, channel, slot, or the like to allow the elongate delivery wire 152 to freely slide through.

According to some embodiments of the disclosure, the first coupler 158 and/or the second coupler 160 may comprise a cylindrical tubular body e.g., in the form of a bushing. The cylindrical tubular body or bushing may have an outer surface configured for contacting the tubular implant 120 and a lumen allowing the delivery wire 152 to freely pass through. The outer and inner diameters of the cylindrical bushing can be selected such that when the tubular implant 120 and the bushings are constrained in the lumen 104 of the catheter 102, the friction force between the compressed bushing and the delivery wire 152 is sufficient to prevent free rotation of the bushing relative to the delivery wire 152. As such, the implant 120, the distal coupler 158 and the proximal coupler 160, and the delivery wire 152 are rotationally coupled together in the lumen 104 of the catheter 102, allowing the features or components on the delivery device 150 to maintain the same position or orientation relative to each other until the first coupler 158 exits the catheter 102. After the first coupler 158 has exited the tip of the catheter 102, the delivery wire 152 can be rotated independently to allow for some control of the delivery device 150 during the implant deployment. After exiting the catheter 102, the bushing of the first coupler 158 can freely rotate about the delivery wire 102. The range of linear movement of the bushing of the first coupler 158 would be limited by the stoppers 154a, 154b of the first set affixed on the delivery wire 152.

According to alternative embodiments of the disclosure, the first coupler 158 can be affixed to the delivery wire 152. This can help prevent relative motion between the implant 120 and the delivery wire 152 in the lumen 104 of the catheter 102 which otherwise would cause twisting of the distal end portion 122 of the implant 120 or of an implant cover. Once the distal end 122 of the implant 120 exits the catheter 102, the contact or gripping between the distal end portion 122 of the implant 120 and the first coupler 158 is undone, allowing the user to torque the delivery wire 152 or control the device 150 independently.

The first coupler 158 has a proximal side or surface configured to engage the proximal stopper 154b of the first set when the delivery wire 152 is pushed to advance the implant 120 constrained in the lumen 104 of the catheter 102. As described above in connection with the first set of stoppers 154a, 154b, the first coupler 158 may comprise a proximal planar or curved surface, or a two- or three-dimensional shape or configuration, configured to engage the proximal stopper 154b of the first set when the delivery wire 152 is pushed in the distal direction. Additionally, or optionally, the first coupler 158 may comprise a distal planar or curved surface, or a two- or three-dimensional shape or configuration, configured to engage the distal stopper 154a of the first set when the delivery wire 152 is retracted in the proximal direction.

The second coupler 160 has a distal side or surface configured to engage the distal stopper 156a of the second set when the delivery wire 152 is pulled to retract the implant 120 in the lumen 104 of the catheter 102. As described above in connection with the second set of stoppers 156a, 156b, the second coupler 160 may comprise a distal planar or curved surface, or a two- or three-dimensional shape or configuration, configured to engage the distal stopper 156a of the second set when the delivery wire 152 is pulled in the proximal direction. Additionally, or optionally, the second coupler 160 may comprise a proximal planar or curved surface, or a two- or three-dimensional shape or configuration, configured to engage the proximal stopper 156b of the second set when the delivery wire 152 is pushed in the distal direction.

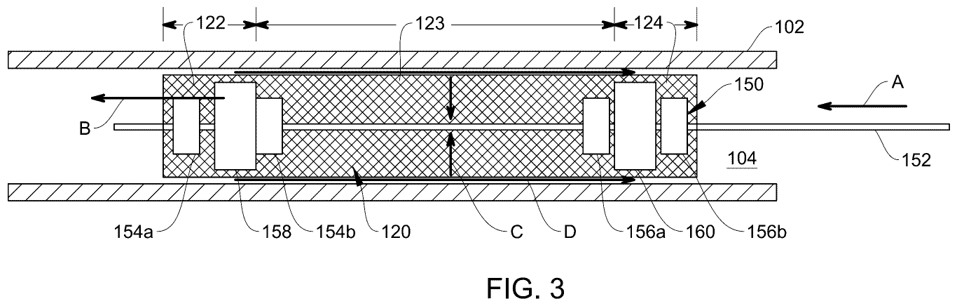

FIG. 3 is a schematic illustrating an operation of a delivery device 150 to advance a braided stent 120 constrained in the lumen 104 of a catheter 102 according to embodiments of the disclosure. To advance the stent 120, the delivery wire 152 can be pushed in a distal direction as indicated by Arrow A. The stoppers 154a, 154b of the first set and the stoppers 156a, 156b of the second set, which are affixed to the delivery wire 152, also move forward as the delivery wire 152 is pushed in the distal direction. According to embodiments of the disclosure, the first coupler 158 disposed between the first set of stoppers 154a, 154b and the second coupler 160 disposed between the second set of stoppers 156a, 156b are arranged such that when the delivery wire 152 is pushed in a distal direction to advance the stent 120, the proximal stopper 154b of the first set engages the first coupler 158, as shown in FIG. 3. As such, a forward force is applied to the first coupler 158 by the proximal stopper 154b of the first set. The grip between the first coupler 158 and the braided stent 120 generates a pulling force, as indicated by Arrow B, which pulls or stretches a portion 123 of the stent 120 proximal of the distal end portion 122 of the stent 120 e.g., the portion 123 between the first coupler 158 and the second coupler 160. As a result, the diameter of the stent portion 130 is reduced as the stent 120 is pulled or stretched, as indicated by Arrow C. The contraction of the stent diameter reduces the normal force and/or surface area of the stent portion 123 against the inner surface of the catheter 102, thereby reducing the overall friction force (indicated by Arrow D) between the stent 120 and the catheter 102. The slight elongation of the stent portion 123 also allows the proximal stopper 156b of the second set to engage the second coupler 160, allowing a forward force to be applied to the second coupler 160. Collectively, the forward force applied by the proximal stopper 154b of the first set, the pulling force in the distal direction generated by the first coupler 158 on the stent portion 123, and the forward force applied by the distal stopper 156b of the second set allow the stent 120 constrained in the lumen 104 of the catheter 102 to overcome the opposing friction and move forward. The pulling force in the distal direction generated by the first coupler 158 on the stent portion 123 reduces the overall resistance in advancing the stent 120 constrained in the lumen 104 of the catheter 102.

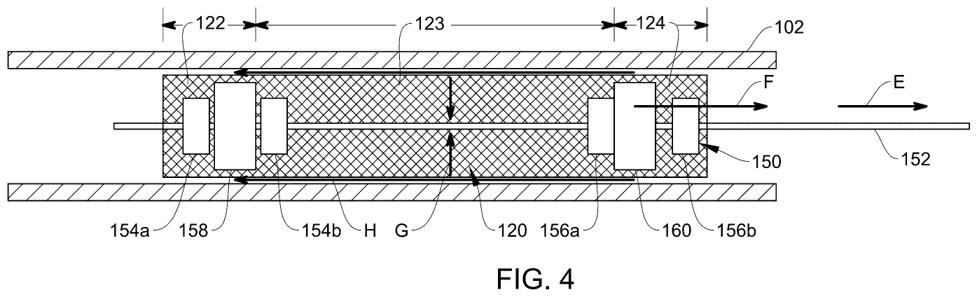

FIG. 4 is a schematic illustrating an operation of a delivery device 150 to retract a braided stent 120 constrained in the lumen 104 of a catheter 102 according to embodiments of the disclosure. To retract the stent 120, the delivery wire 152 can be pulled in a proximal direction as indicated by Arrow E. The stoppers 154a, 154b of the first set and the stoppers 156a, 156b of the second set, which are affixed to the delivery wire 152, also move rearward as the delivery wire 152 is pulled in the proximal direction. According to embodiments of the disclosure, the first coupler 158 disposed between the first set of stoppers 154a, 154b and the second coupler 160 disposed between the second set of stoppers 156a, 156b are arranged such that when the delivery wire 152 is pulled in a proximal direction to retract the stent 120, the distal stopper 156a of the second set engages the second coupler 160, as shown in FIG. 4. As such, a rearward force is applied to the second coupler 160 by the distal stopper 156a of the second set. The grip between the second coupler 160 and the braided stent 120 generates a pulling force, as indicated by Arrow F, which pulls or stretches a portion 123 of the stent 120 distal of the proximal end portion 124 of the stent 120 e.g., the portion 123 between the first coupler 158 and the second coupler 160. As a result, the diameter of the stent portion 123 is reduced as the stent 120 is pulled or stretched, as indicated at Arrow G. The contraction of the stent diameter reduces the normal force and/or surface area of the stent portion 123 against the inner surface of the catheter 102, thereby reducing the overall friction force (indicated by Arrow H) between the stent 120 and the catheter 102. The slight elongation of the stent portion 123 also allows the distal stopper 154a of the first set to engage the first coupler 158, allowing a rearward force to be applied to the first coupler 158. Collectively, the rearward force applied by the distal stopper 156a of the second set, the pulling force in the proximal direction generated by the second coupler 160 on the stent portion 123, and the rearward force applied by the distal stopper 154a of the first set allow the stent 120 constrained in the lumen 104 of the catheter 102 to overcome the opposing friction and move rearward. The pulling force in the proximal direction generated by the second coupler 160 on the stent portion 123 reduces the overall resistance in retracting the stent 120 constrained in the lumen 104 of the catheter 102.

Therefore, according to embodiments of the disclosure the position of the proximal stopper 154b of the first set with respect to the first coupler 158 and the position of the proximal stopper 156b of the second set with respect to the second coupler 160 can be arranged to allow the proximal stopper 154b of the first set to engage the first coupler 158 before the proximal stopper 156b of the second set engages the second coupler 160 when the delivery wire 152 is pushed in a distal direction to advance an implant 120. As such, a pulling force in the distal direction can be generated by the first coupler 158 on an implant portion 123 between the first coupler 158 and the second coupler 160. The pulling force induces slight diametrical contraction of the implant portion 123, thereby reducing the overall static friction between the implant 120 and the catheter 102. The elongation of the implant portion 123 induced by the pulling force also allows the proximal stopper 156b of the second set to engage the second coupler 160, allowing a pushing force to be applied to the second coupler 160 to advance the implant 120.

Conversely, according to embodiments of the disclosure the position of the distal stopper 154a of the first set with respect to the first coupler 158 and the position of the distal stopper 156a of the second set with respect to the second coupler 160 can be arranged to allow the distal stopper 156a of the second set to engage the second coupler 160 before the distal stopper 154a of the first set engages the first coupler 158 when the delivery wire 152 is pulled in a proximal direction to retract an implant 1020. As such, a pulling force in the proximal direction can be generated by the second coupler 160 on an implant portion 123 between the first coupler 158 and the second coupler 160. The pulling force induces slight diametrical contraction of the implant portion 123 between the first coupler 158 and the second coupler 160, thereby reducing the overall static friction between the implant 120 and the catheter 102. The elongation of the implant 120 induced by the pulling force also allows the distal stopper 154b of the first set to engage the first coupler 158, allowing a force to be applied to the first coupler 158 to retract the implant 120.

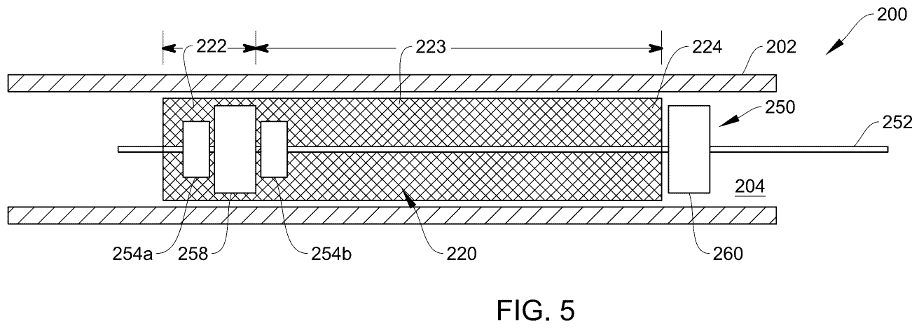

It should be noted that while various embodiments are described in conjunction with two couplers and two sets of stoppers as shown in FIGS. 2-4, a delivery device according to embodiments of the disclosure may include less or more than two couplers and less or more than two sets of stoppers. FIG. 5 depicts an endovascular system 200 comprising a delivery device 250 having one coupler and one set of stoppers according to alternative embodiments of the disclosure.

As shown in FIG. 5, the example endovascular system 200 comprises an elongate tubular member or catheter 202 having a lumen 204, a tubular implant 220 disposed in the lumen 204 of the catheter 202, and a delivery device 250 operable to deliver the tubular implant 220 to a target site. The delivery device 250 can generate a pulling force in the distal direction on the tubular implant 220 to aid advancement of the implant 220.

The catheter 202 and the tubular implant 220 can be the same as or resembles the catheter 102 and the tubular implant 120 described above in conjunction with FIGS. 1-4. In comparison, the delivery device 250 shown in FIG. 5 comprises a delivery wire 252, a set of stoppers 254a, 254b, a coupling feature or coupler 258, and a bumping feature or bumper 260. The coupler 258 is disposed between the stoppers 254a, 254b and configured to contact a distal end portion 222 of the tubular implant 220 and apply an outwardly radial force to grip the tubular implant 220 in the lumen 204 of the catheter 202. The bumper 260 is disposed adjacent to the proximal end 224 of the implant 220 and configured to apply a forward or rearward force to the implant 220 at the proximal end 224 of the implant 220.

With reference to FIG. 5, the set of stoppers 254a, 254b can comprise a stopper 254a distal of the coupler 258 and a stopper 254b proximal of the coupler 258. The distal stopper 254a and the proximal stopper 254b can be affixed to the delivery wire 252 and thus can be advanced, retracted, and rotated with the delivery wire 252. The delivery wire 252, the distal stopper 254a, the proximal stopper 254b, and the coupler 258 can be the same as or resembles the delivery wire 152, the first set of stoppers 154a, 154b, and the first coupler 158 described above in conjunction with FIGS. 2-4. The bumper 160 adjacent to the proximal end 224 of the tubular implant 220 can be coupled to the delivery wire 252 in any suitable manner. The bumper 260 can be constructed from any suitable material such as metals, metal alloys, polymers, or any combination thereof. The bumper 260 can be in any suitable form or configuration such as tube, cone, cylinder, ellipsoid, or the like.

To advance the tubular implant 220, the delivery wire 252 can be pushed in a distal direction. The stoppers 254a, 254b, which are affixed to the delivery wire 252, also move forward as the delivery wire 252 is pushed in the distal direction. According to embodiments of the disclosure, the coupler 258, the stoppers 254a, 254b, and the proximal bumper 260 are arranged such that when the delivery wire 252 is pushed forward, the proximal stopper 254b engages the coupler 258. As such, a forward force is applied to the coupler 258 by the proximal stopper 254b. The grip between the coupler 258 and the implant 220 slightly pulls or stretches a portion 223 of the implant 220 proximal of the distal end portion 222 of the implant 220. As a result, the diameter of the implant portion 223 is reduced as the implant 220 is pulled or stretched. The contraction of the implant diameter reduces the normal force and/or surface area of the implant portion 223 against the inner surface of the catheter 202, thereby reducing the overall friction between the implant 220 and the catheter 202. Collectively, the forward force applied by the proximal stopper 254b, the pull force in the distal direction generated by the coupler 258 on the implant portion 223, and a forward force applied by the bumper 260 allow the implant 220 constrained in the lumen 204 of the catheter 202 to overcome the opposing friction and move forward. The pulling force in the distal direction generated by the coupler 258 on the implant portion 223 reduces the overall resistance in advancing the implant 220 constrained in the lumen 204 of the catheter 202. To retract the implant 220, the delivery wire 252 can be pulled in a proximal direction. The distal stopper 254a affixed to the delivery wire 252 can apply a rearward force to the coupler 258 to assist retraction of the implant 220 into the catheter 202.

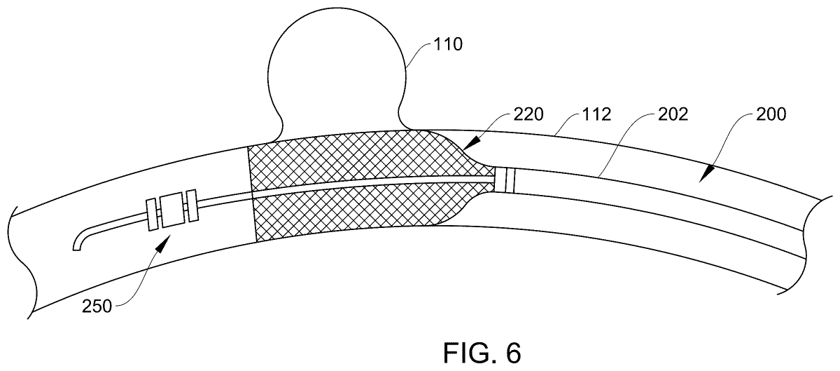

FIG. 6 illustrates an application of an example endovascular system 200 of the disclosure to treat an aneurysm 110 in a cerebral vasculature 112. In use, a tubular implant 220 such as a braided flow diverting stent can be loaded on the delivery device 250 of the disclosure inside a microcatheter 202. The flow diverting stent 220 and the delivery device 250 within the microcatheter 202 can be introduced to the target site through an access e.g., in the femoral artery or groin area of the patient by using an introducer sheath or guiding catheter (not shown). The endovascular system 200 can be guided to the target site through a guidewire (not shown). The guidewire can be visible via fluoroscopy, allowing the endovascular system to be reliably advanced over the guidewire to the target site.

Once the target site has been accessed, the guidewire can be withdrawn. The flow diverting stent 220 can be then delivered using the delivery device 250 of the disclosure as described above in connection with FIGS. 2-5. The physician may advance and retract the stent 220 several times to obtain a desirable position of the stent 220 relative to the aneurysm 110 neck before a complete release of the stent 220. Once the stent 220 is satisfactorily positioned, the physician may push the delivery wire 252 distally allowing the stent 220 to fully exit the microcatheter 202 and expand in a deployed configuration at the target site. The delivery device 250 can be then withdrawn into the microcatheter 202 and removed out of the patient.

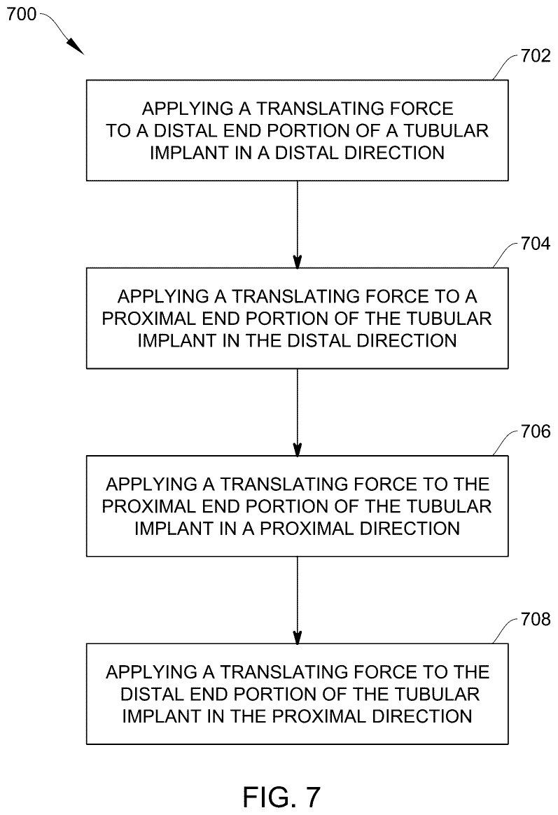

With reference now to FIG. 7, a method 700 according to embodiments of the disclosure is described. The method 700 can be used to deliver a tubular implant such as a braided tubular stent to a cerebral vasculature or other treatment sites in a patient. The method 700 can be performed using an endovascular system 100, 200 of the disclosure described in conjunction with FIGS. 1-5 or any other suitable systems.

The method 700 may begin by introducing an endovascular system to a treatment site in a patient such as a cerebral vasculature. The endovascular system may comprise an elongate tubular member such as a catheter or sheath, a tubular implant disposed or constrained in the lumen of the catheter, and a delivery device operable to advance the tubular implant relative to the catheter for deployment of the implant and/or to retract the tubular implant for repositioning.

At step 702, a translating force in a distal direction is applied to a distal end portion of the tubular implant. The translating force in the distal direction to the distal end portion of the implant can be applied by the delivery device using a coupling feature or coupler (distal coupler), which contacts the distal end portion of the tubular implant in the catheter and applies an outwardly radial force to grip the implant in the lumen of the catheter. A stopper affixed to a delivery wire can be used to push the distal coupler from the proximal side of the distal coupler. The distal coupler can then transfer the translating force in the distal direction to the distal end portion of the implant. Because of the translating force in the distal direction applied to the distal end portion of the implant, the portion of the implant proximal to the distal end portion of the implant is pulled or stretched, reducing the diameter of the implant portion. The contraction of the implant diameter reduces the normal force and/or surface area of the implant portion against the inner surface of the catheter, thereby reducing the overall friction between the implant and the catheter.

At step 704, a translating force in a distal direction is applied to a proximal end portion of the tubular implant. The translating force in the distal direction to the proximal end portion of the implant can be applied by the delivery device using a coupling feature or coupler (proximal coupler), which contacts the proximal end portion of the tubular implant in the catheter and applies an outwardly radial force to grip the implant in the lumen of the catheter. A stopper affixed to a delivery wire can be used to push the proximal coupler from the proximal side of the proximal coupler. The proximal coupler can then transfer the translating force in the distal direction to the proximal end portion of the implant. Collectively, the translating force applied by a stopper to the distal coupler, the pulling force in the distal direction generated by the distal coupler on the implant portion, and the translating force applied by a stopper to the proximal coupler allow the implant constrained in the lumen of the catheter to overcome the opposing friction and move forward. The pulling force in the distal direction generated by the distal coupler on the implant portion reduces the overall resistance in advancing the implant constrained in the lumen of the catheter.

According to embodiments of the disclosure, the translating force in the distal direction to the distal end portion of the tubular implant (Step 702) is applied before the applying of the translating force in the distal direction to the proximal end portion of the tubular implant (Step 704). This can be accomplished by arranging the positions of the stoppers affixed on the delivery wire with respect to the distal coupler and the proximal coupler such that when the delivery wire is pushed in the distal direction, a stopper engages the distal coupler first before a stopper engages the proximal coupler.

According to embodiments of the disclosure, the method may further comprise retracting of the implant in a proximal direction. Retraction or resheathing of the implant into the catheter may be needed as determined by the physician for repositioning of the implant before complete release of the implant. The retracting or re-sheathing of the implant may include step 706 and step 708.

At step 706, a translating force in a proximal direction is applied to the proximal end portion of the tubular implant. The translating force in the proximal direction to the proximal end portion of the implant can be applied by the delivery device using the proximal coupler, which contacts the proximal end portion of the tubular implant in the catheter and applies an outwardly radial force to grip the implant in the lumen of the catheter. A stopper affixed to a delivery wire can be used to push the proximal coupler from the distal side of the proximal coupler. The proximal coupler can then transfer the translating force in the proximal direction to the proximal end portion of the implant. Because of the translating force in the proximal direction applied to the proximal end portion of the implant, the portion of the implant distal of the proximal end portion of the implant is pulled or stretched, reducing the diameter of the implant portion. The contraction of the implant diameter reduces the normal force and/or surface area of the implant portion against the inner surface of the catheter, thereby reducing the overall friction between the implant and the catheter.

At step 708, a translating force in a proximal direction can be applied to a distal end portion of the tubular implant. The translating force in the proximal direction to the distal end portion of the implant can be applied by the delivery device using the distal coupler, which contacts the distal end portion of the tubular implant in the catheter and applies an outwardly radial force to grip the implant in the lumen of the catheter. A stopper affixed to a delivery wire can be used to push the distal coupler from the distal side of the distal coupler. The distal coupler can then transfer the translating force in the proximal direction to the distal end portion of the implant. Collectively, the translating force applied by a stopper to the proximal coupler, the pulling force in the proximal direction generated by the proximal coupler on the implant portion, and the translating force applied by a stopper to the distal coupler allow the implant constrained in the lumen of the catheter to overcome the opposing fricition and move rearward. The pulling force in the proximal direction generated by the proximal coupler on the implant portion reduces the overall resistance in retracting or resheathing the implant constrained in the lumen of the catheter.

According to embodiments of the disclosure, the translating force in the proximal direction to the proximal end portion of the tubular implant (Step 706) is applied before the applying of the translating force in the proximal direction to the distal end portion of the tubular implant (Step 708). This can be accomplished by arranging the positions of the stoppers affixed on the delivery wire with respect to the proximal coupler and the distal coupler such that when the delivery wire is pulled in the proximal direction, a stopper engages the proximal coupler first before a stopper engages the distal coupler.

Various embodiments of an endovascular system including a delivery device have been described. Advantageously, the delivery device of the disclosure comprises one or more coupling features to contact and/or grip a tubular implant constrained in the lumen of a catheter, which can generate a pulling force on the implant in both advancement and retraction direction, thereby reducing the overall delivery forces. This is particularly advantageous in endovascular treatment of cerebrovascular diseases such as aneurysms. Flow diverting stents are commonly used to treat cerebral aneurysms. However, the delivery of a flow diverting stent in a cerebral anatomy especially in small distal cerebral vessels presents challenges. To deliver a flow diverting stent to a cerebral vessel, microcatheters having small diameters are required. For instance, to reach further distal cerebral anatomies, a microcatheter used for delivering a flow diverting stent may have an inner diameter as small as 0.027″, 0.021″, or 0.017″. The flow diverting stent also must be designed for deliverability through the microcatheter and the distal cerebral vessel. To fit in the microcatheter, the size of the flow diverting stent must be reduced, which may affect the pore densities that a stent can achieve. The coupling feature of the disclosure can advantageously reduce the total delivery force, thus improving the deliverability of the stent with a microcatheter. It also allows the flow diverting stent to be constructed without compromising its diameter, radial force, and pore density. To increase stent radial force and pore density, more wires or increased wire diameters are generally used in the stent design. However, in more distal anatomies of the patient, smaller microcatheters are required to navigate, access, or fit in smaller vessels, thus limiting the lumen size that a stent can be delivered through. A smaller microcatheter with a tighter lumen size requires a higher delivery force to deliver a stent to the distal anatomy. Therefore, to reduce delivery forces the wire count or size is typically reduced in the stent design to decrease the stent profile in the microcatheter lumen, which compromises the radial force and pore density of the stent. The coupling feature of the disclosure can advantageously reduce the delivery forces on the delivery system without having to compromise as much on the stent design for wire count and size.

Another advantage of the disclosure is that the distal coupler can serve as a back-up coupling feature in case the proximal coupler loses contact of the implant during resheath. On rare occasions, a braided stent can lose contact with its proximal coupler during resheathing. With a distal coupling feature, the delivery system can be withdrawn further to allow the distal coupler to re-engage contact with the remaining segment of implant constrained in the catheter and regain the ability to move the implant. A further advantage of having a distal coupling feature is the ability to improve the distal opening of the implant. For braided stents with exposed wire ends delivered via an empty catheter, the distal ends of the stent need be protected from friction or damage with a covering feature, which can impede good stent opening. A relatively larger profile of the distal coupler and its proximity to the distal end of the constrained device can help ensure that the braided wires are more biased to expand fully.

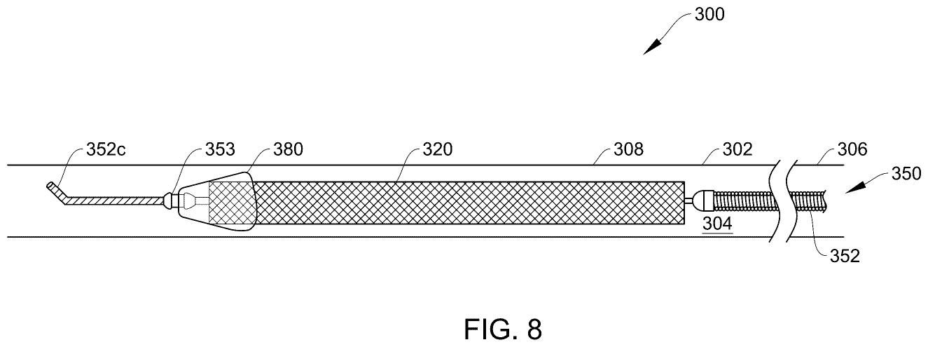

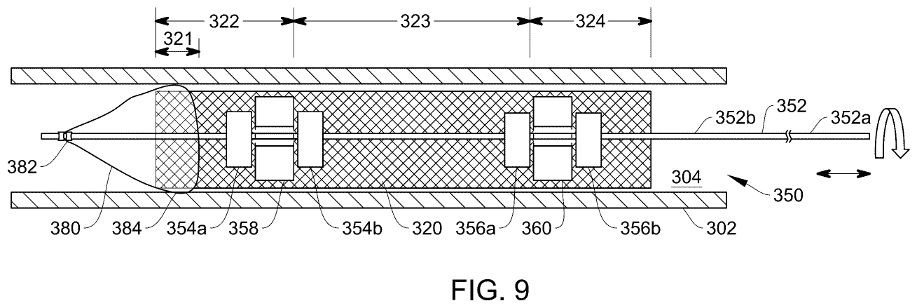

With reference now to FIGS. 8-9, an endovascular system 300 according to alternative embodiments of the disclosure is now described. In a broad overview, the example endovascular system 300 comprises a catheter 302, a tubular implant 320, and a delivery device 350 operable to deliver and deploy the tubular implant 320. The delivery device 350 comprises a protection cover 380 wrapping around at least an end portion of the tubular implant 320 to protect the implant from damage during delivery and deployment of the implant.

In conventional stent delivery, if a stent, after exiting a delivery catheter, is still restrained by a protection cover thus preventing the stent from opening or expanding, the stent is typically re-sheathed or withdrawn into the catheter by relative movement of the catheter and a delivery wire. However, resheathing a tubular stent into a catheter can be difficult to accomplish, especially in tortuous anatomy such as the cerebral vasculature. Another issue of conventional delivery is that during the deployment of the stent, the delivery wire may get caught on a tight vessel bend or may go into a perforator, a small artery branching off the main artery. As a result, the delivery wire can no longer advance forward, preventing deployment of the stent. If such incidence occurs, the physician has to resheath and redeploy the stent, hoping that the delivery wire tip takes a different orientation and the problem does not reoccur during the deployment. The inability to remove a protection cover even via resheathing is common in the conventional stent delivery, and the occurrence rate can be as high as more than 25 percent of the time. According to embodiments of the disclosure, an endovascular system allows relative rotational movement of a protection cover and a tubular implant to thereby facilitate release and deployment of the implant. A delivery device of the disclosure includes a torqueable delivery wire with a shaped tip segment to aid the physician to navigate through tortuous geometry and avoid the risk of perforating the vessel wall or getting stuck in an artery branching off the main artery.

With reference to FIGS. 8-9, the example endovascular system 300 according to embodiments of the disclosure comprises a catheter 302 having a lumen 304, a tubular implant 320, and a delivery device 350 operable to deliver the tubular implant 320 through the catheter 302. The tubular implant 320 has a collapsed state for being disposed in the lumen 304 of the catheter 302 and an expanded state when unconstrained by the catheter 302. The delivery device 350 generally includes an elongate delivery wire 352 having a proximal section 352a and a distal section 352b, one or more sets of stoppers 354a/354b, 356a/356b fixedly attached to the distal section 352b of the delivery wire 352, one or more couplers 358, 360 disposed between the one or more sets of stoppers 354a/354b, 356a/356b respectively (FIG. 9), and a protection cover 380. The one or more couplers 358, 360 are configured to contact and apply an outwardly radial force to a portion of the tubular implant 320 to grip the implant in the lumen 304 of the catheter 302. The one or more couplers 358, 360 are provided with a through-opening allowing the delivery wire 352 to pass through. The protection cover 380 includes a first end portion 382 fixedly coupled to the distal section 352b of the delivery wire 352 and a second end 384 wrapping at least an end portion of the tubular implant 320 in the lumen 304 of the catheter 302. As will be described in greater detail below, the one or more couplers 358, 360 are configured to allow the distal section 352b of the delivery wire 352 to slide longitudinally and rotate relative to the one or more couplers 358, 360. The longitudinal movement of the delivery wire 352 in the lumen 304 of the catheter 302 allows the one or more stoppers 354a/354b, 356a/356b to engage the one or more couplers 358, 360 for advancing or withdrawing the tubular implant 320. The rotation of the delivery wire 302 relative to the one or more couplers 358, 360 allows the protection cover 380 to pull off from the end portion of the tubular implant 320 when exiting the lumen 304 of the catheter 302 to thereby facilitate opening or expanding of the tubular implant 320.

With reference to FIGS. 8-9, the catheter 302 can be any suitable tubular member in the form of a sheath, catheter, microcatheter, or other suitable tubular forms. The catheter 302 may include a proximal end portion 306, a distal portion 308, and a lumen 304 extending between the proximal portion 306 and the distal portion 308. The proximal portion 306 may remain outside of the patient and is accessible to the user or physician when the endovascular system is in use. The distal portion 308 may be sized and dimensioned to reach a remote location in the vasculature of the patient, such as in a cerebral vessel adjacent to an aneurysm, a bifurcated blood vessel, an occlusion in a blood vessel, or the like. The lumen 304 of the catheter 302 can be sized to accommodate the tubular implant 320 and the delivery device 350 including the stoppers 354, 356 and the couplers 358, 360. While not explicitly shown, the catheter 302 may include one or more sections or regions each of which may have different configurations and/or characteristics. For example, the distal portion 308 of the catheter 302 may include a flexible section or region comprising a coil to provide proper bending or deflection. A flexible distal portion would allow the endovascular system to navigate more easily through tortuous regions of the vasculature to remote locations in the patient. The proximal portion 306 may be constructed from a stiffer material e.g., a rigid metal hypotube, to provide structural stability and sufficient pushability. In general, the catheter 302 or a section of the catheter 302 can be constructed of suitable biocompatible polymers, metals, or combinations thereof. The distal portion 308 of the catheter 302 can have an outer diameter less than the outer diameter of the proximal portion 306 to reduce the profile of the distal portion and facilitate navigation through tortuous vasculature. While not shown, the catheter 302 may include one or more markers which can be viewed e.g., via fluoroscopy, to assist the physician in operation of the endovascular system. The inner and outer diameters of the catheter 302 at the distal portion 308 can be properly chosen based on applications. By way of example, for application of treating cerebral aneurysms, the catheter or microcatheter 302 may have an inner diameter ranging from 0.0165 inches to 0.040 inches for delivering a flow diverting stent of a suitable size to a target site in the cerebral anatomies or distal cerebral vessels.

With reference to FIGS. 8-9, the tubular implant 320 can be any suitable implant compatible with the delivery device of the disclosure. By way of example, the tubular implant 320 can be an embolic device such as a stent, flow diverting stent, or an intrasaccular device for treatment of brain aneurysms. The term “tubular implant” may be used interchangeably with the term “stent” in describing various embodiments of the disclosure. The tubular implant 320 can be expandable, having a collapsed state when compressed or constrained in the lumen of the catheter, and an expanded state when exiting the catheter unconstrained or deployed at a treatment site. As shown, the tubular implant 320 comprises a distal portion 322 a proximal portion 324, and a mid-portion 323 between the distal portion 322 and the proximal portion 324.

The tubular implant 320 can comprise a braided structure or a patterned cut tube structure. The braided or patterned cut tube structure can be a closed-cell design, in which the repeated ring structure is connected at all strut junctions. The braided or patterned cut tube structure can also be an open-cell stent design, in which some junctions between the repeated ring structure are removed.

An example tubular implant 320 comprises a braided stent constructed of a plurality of wires or filaments. The plurality of filaments can be braided, woven, or interlaced in a suitable pattern. By way of example, a plurality of filaments can extend spirally or helically clockwise and a plurality of filaments extend spirally or helically counterclockwise, forming a plurality of cross sections and defining a plurality of cells of a radially expandable body. The braided stent can be a closed-cell design. The filaments constructing the braided stent can be metallic or polymeric. The filaments can be radiopaque or non-radiopaque, or at least one or more of the filaments making up the stent are radiopaque. Depending on application, a braided stent may comprise about 40 to about 96 filaments. The filaments may have a diameter ranging from 0.0008 to 0.0030 inches. The filaments can have a shape-memory property and/or can be heat set to form a self-expanding stent. In an expanded state, the braided stent may have a maximal diameter ranging from 1.0 mm to 10 mm depending on applications. For treatment of cerebral aneurysms, a braided stent can be constructed as a flow diverting device having a pore size and/or density suitable to disrupt or divert blood flow near an aneurysm neck, resulting in occlusion of the aneurysm while allowing blood flow in the parent and branch vessels.

According to embodiments of the disclosure, at least some of the filaments making the tubular implant 320 are composite wires such as drawn-filled tubing (DFT) wires. A composite wire e.g., DFT wire comprises an inner core of a first material and an outer sheath of a second material different from the first material.

Suitable materials for the inner core of the composite wires can be materials having relative stiff, crush resistant properties. Example materials for the inner core of the composite wires include but are not limited to nickel-cobalt alloys (e.g., MP35N), stainless steel, cobalt-chromium alloy, and so on. A stiff, crush resistant inner core material can provide a tubular implant, when disposed within the lumen of a delivery catheter, with a greater radial force against the inner surface of the delivery catheter. According to alternative embodiments of the disclosure, the inner core of the composite wires can be a radiopaque material visible via fluoroscopy, including but not limited to tungsten, platinum, iridium, gold, tantalum, or any alloy thereof such as a platinum-iridium alloy, a platinum-tungsten alloy, and so on.

Suitable materials for the outer sheath of the composite wires can be materials having elastic or hyperelastic properties. Example materials for the outer sheath of the composite wires include but are not limited to nickel-titanium alloys (e.g., Nitinol). An elastic or hyperelastic outer sheath material can provide a tubular implant 320 with greater flexibility, lending it to ease of use. According to embodiments of the disclosure, the outer sheath of the composite wires comprises a cobalt-chromium alloy, which can provide structural strength to the DFT wires leading to a braided stent with higher radial force.

According to embodiments of the disclosure, the fill percentage of the inner core of the composite wires can be chosen to provide a desired balance of flexibility and radial resistance for the tubular implant 320. As used herein, the phrase “fill percentage” refers to the proportion of the composite wire's cross-sectional area occupied by the inner core material (filled material) relative to the total cross-sectional area of the composite wire. According to embodiments of the disclosure, the composite wires comprise a fill percentage of the inner core material ranging from about 5 to 35 percent, or 5 to 30 percent, or 10 to 30 percent. In some embodiment, the composite wires comprise a fill percentage of the inner core material no greater than 30 percent.

According to embodiments of the disclosure, the tubular implant 320 comprises a braided stent constructed of a plurality of composite wires comprising an inner core of a nickel-cobalt alloy (e.g., MP35N) and an outer sheath of a nickel-titanium alloy (e.g., Nitinol). The composite wires comprise a fill percentage of the nickel-cobalt alloy inner core ranging from about 5 to 30 percent.

Advantageously, the tubular implant 320 constructed of composite wires according to embodiments of the disclosure can both maintain the hyperelasticity of the implant offered by e.g., pure Nitinol wires, and achieve higher radial forces and radial resistive forces offered by e.g., MP35N. For example, a braided stent 320 of the disclosure constructed of MP35N-Nitinol composite wires or DFT wires can achieve a blend of high radial force, stiffness, and crush resistance offered by cobalt-chromium implants, and hyperelasticity and ease of use offered by Nitinol implants, thereby solving the dilemma of having to choose between cobalt chromium implants and Nitinol stents as experienced in the prior art. Another advantage is that it allows the use of a smaller diameter of composite wires to achieve the same radial force provided by e.g., pure Nitinol wires of a greater diameter, which in turn allows use of a delivery catheter of a smaller diameter for delivery of the implant. This is highly desirable in some areas such as neuro vasculature.

With reference to FIGS. 8-9, the delivery device 350 comprises an elongate delivery wire 352, one or more sets of stoppers 354a/354b, 356a/356b fixedly attached to the delivery wire 352, one or more couplers 358, 360 disposed between the one or more sets of stoppers 354a/354b, 356a/356b respectively, and a protection cover 380. FIG. 9 depicts an example delivery device 350 comprising a first set of stoppers 354a, 354b affixed to the delivery wire 352, a first coupler 358 disposed between the first set of stoppers 354a, 354b, a second set of stoppers 356a, 356b affixed to the delivery wire 352, and a second coupler 360 disposed between the second set of stoppers 354a, 354b. It should be noted that the delivery device 350 can include less or more than two sets of stoppers and less or more than two couplers.

With reference to FIGS. 8-9, the elongate delivery wire 352 has a proximal section 352a, a distal section 352b, and a length extending between the proximal section 352a and the distal section 352b. The proximal section 352a and distal section 352b of the delivery wire 352 may have difference profiles, configurations, and/or comprise different materials. For instance, the proximal section 352a may be constructed from a stiffer material e.g., a rigid metal hypotube, and/or have a greater diameter to provide structural stability and sufficient pushability. The distal section 352b of the delivery wire may be more flexible or have a reduced profile to provide proper bending or deflection for navigating through tortuous vasculature or reach remote small vessels. The proximal section 352a of the delivery wire 352 may remain outside of the patient and is accessible to the physician when the delivery device is in use. The proximal section 352a, which may be coupled to a handle, can be controlled by the user in operation. For example, the proximal section 352a may be pushed, pulled, and/or twisted, to advance or withdraw the tubular implant 320 through the catheter 302, and/or remove the protection cover 380 off from the end portion of the tubular implant 320 after exiting the catheter 302, to be described in greater detail below. The one or more sets of stoppers 354a/354b, 356a/356b can be fixedly coupled to the distal section 352b of the delivery wire 352, and therefore can be pushed, pulled, and and/or rotated with the delivery wire 352. The delivery wire 352 may include one or more markers e.g., a marker 353 on the distal section of the delivery wire 352, to aid the physician in operating the delivery device 350 via fluoroscopy. The delivery wire 152 may be constructed from a suitable metal such as stainless steel, nickel, titanium, nitinol, an alloy of metals, a biocompatible polymer, a shape memory polymer, hypotube, or any combinations thereof.

With reference to FIGS. 8-9, the delivery wire 352 may include a shaped tip segment 352c. By way of example, the tip segment 352c of the delivery wire 352 can be heat-set or processed to have an angled, curved, or other shaped configuration, and retain the shaped configuration. For example, an angled tip segment 352c may extend distally and be oriented to bend or angle from the longitudinal axis of the delivery wire 352. The angled tip segment 352c may form an angle ranging from 15-90 degrees with the longitudinal axis of the delivery wire 352. One advantage of a shaped tip segment is that as the deliver wire 352 is torqued at the proximal section 352a, the orientation of the shaped tip segment 352c changes, allowing the physician to navigate through tortuous geometry, avoid the entrance to branching arteries, and/or avoid the risk of perforating the vessel wall.

With reference to FIGS. 8-9, the one or more sets of stoppers 354a/354b, 356a/356b can be fixedly coupled to the delivery wire 352. For example, a first set of stoppers 354a, 354b and a second set of stoppers 356a, 356b can be affixed to the distal section 352b of the delivery wire 352. The locations of the first set of stoppers 354a, 354b and the second set of stoppers 356a, 356b can be chosen such that the first coupler 358 disposed between the first set of stoppers 354a, 354b can be located adjacent to the distal portion 322 of the tubular implant 320 and thereby apply an outwardly radial force to the tubular implant 320 to grip the implant in the lumen 304 of the catheter 302, and the second coupler 360 disposed between the second set of stoppers 356a, 356b can be located adjacent to the proximal portion 324 of the tubular implant 320 and thereby apply an outwardly radial force to the tubular implant 320 to grip the implant 320 in the lumen 304 of the catheter 302. As will be described in greater detail below, the arrangement of the first set stoppers 354a, 354b and the first coupler 358 allows the first coupler 358, which grips the distal portion 322 of the tubular implant 320, to generate a pulling force in the distal direction on the portion 323 of the implant proximal of the distal portion 322 of the implant 320, thus aiding advancement of the implant 320 out of the catheter 302. The arrangement of the second set stoppers 356a, 356b and the second coupler 360 allows the second coupler 360, which grips the proximal portion 324 of the tubular implant 320, to generate a pulling force in the proximal direction on the portion 323 of the implant distal of the proximal portion 324 of the implant 320, thus aiding retraction of the implant 320 back into the catheter 302.

In the example shown in FIG. 9, the first set of stoppers 354a, 354b comprises a stopper 354a distal of the first coupler 358 and a stopper 354b proximal of the first coupler 358. The distal stopper 354a and the proximal stopper 354b of the first set can be fixedly attached to the distal section 352b of the delivery wire 352 and thus can be advanced, retracted, and/or rotated with the delivery wire 352. The stoppers of the first set 354a, 354b can be sized such that they do not directly contact the tubular implant 320 in the lumen 304 of the catheter 302, or do not create, via direct contact with the tubular implant 320, a frictional force sufficient to advance or retract the implant 320 constrained in the lumen 304 of the catheter 302. In some embodiments, the stoppers 354a, 354b of the first set are sized such that their cross-section is smaller than the cross section of the first coupler 358.

The proximal stopper 354b of the first set can be configured to engage the first coupler 358 when the delivery wire 352 is advanced to apply a pushing force to the first coupler 358 from the proximal side of the first coupler 358. By way of example, the proximal stopper 354b of the first set may comprise a planar distal surface configured to engage a planar proximal surface of the first coupler. Other configurations and shapes of the engagement surfaces between the proximal stopper 354b of the first set and the first coupler 358 are possible and will be appreciated by one of ordinary skill in the art. These and other configurations and shapes of the engagement surfaces can be planar or curved, two-dimensional, or three-dimensional, and the scope of the disclosure is not limited to any specific configurations or shapes of the engagement surfaces between the proximal stopper of the first set and the first coupler. When the delivery wire 352 is advanced, the proximal stopper 354b of the first set engages the first coupler 358 and applies a pushing force to the first coupler 358 in the distal direction. The force applied by the proximal stopper 354b of the first set can be transferred to the distal portion 322 of the tubular implant 320 gripped by the first coupler 358, thereby generating a pulling force on the portion 323 of the tubular implant 320 proximal of the distal end portion 322 of the implant 320 e.g., the portion of the implant between the first coupler 358 and the second coupler 360. Additionally, or optionally, the distal stopper 354a of the first set can be configured to engage the first coupler 358 when the delivery wire 352 is retracted. The distal stopper 354a of the first set may comprise a proximal surface, either planar or curved, or a configuration and shape, either two-dimensional or three-dimensional, for engaging the first coupler 358. In retracting or re-sheathing the tubular implant 320, the distal stopper 354a of the first set may engage the first coupler 358 and apply a pushing force to the first coupler 358 in the proximal direction from the distal side of the first coupler 358.

With reference to FIG. 9, the second set of stoppers 356a, 356b may comprise a stopper 356a distal of the second coupler 360 and a stopper 356b proximal of the second coupler 360. The distal stopper 356a and the proximal stopper 356b of the second set can be fixedly attached to the delivery wire 352 and thus can be advanced, retracted, and/or rotated with the delivery wire 352. The stoppers 356a, 356b of the second set can be sized such that they do not directly contact the tubular implant 320 in the lumen 304 of the catheter 302, or do not create, via direct contact with the tubular implant 320, a frictional force sufficient to retract or advance the implant 320 constrained in the lumen 304 of the catheter 302. In some embodiments, the stoppers 356a, 356b of the second set are sized such that their cross-section is smaller than the cross section of the second coupler 360.

The distal stopper 356a of the second set can be configured to engage the second coupler 360 when the delivery wire 352 is retracted to apply a pushing force to the second coupler 360 from the distal side of the second coupler 360. By way of example, the distal stopper 356a of the second set may comprise a planar proximal surface configured to engage a planar distal surface of the second coupler 360. Other configurations and shapes of the engagement surfaces between the distal stopper 356a of the second set and the second coupler 360 are possible and will be appreciated by one of ordinary skill in the art. These and other configurations and shapes of the engagement surfaces can be planar, curved, two-dimensional, or three-dimensional, and the scope of the disclosure is not limited to any specific configurations or shapes of the engagement surfaces between the distal stopper of the second and set the second coupler. When the delivery wire 352 is retracted, the distal stopper 356a of the second set engages the second coupler 360 and applies a pushing force to the second coupler 360 in the proximal direction. The force applied by the distal stopper 356a of the second set can be transferred to the proximal portion 324 of the tubular implant 320 gripped by the second coupler 360, thereby generating a pulling force on the portion 323 of the tubular implant 320 distal of the proximal portion 324 of the implant e.g., the portion 323 of the implant between the first coupler 358 and the second coupler 360. Additionally, or optionally, the proximal stopper 356b of the second set can be configured to engage the second coupler 360 when the delivery wire 352 is advanced. The proximal stopper 356b of the second set may comprise a distal surface, either planar or curved, or a configuration and shape, either two-dimensional or three-dimensional, for engaging the second coupler. In advancing the tubular implant 320, the proximal stopper 356b of the second set may engage the second coupler 360 and apply a pushing force to the second coupler 360 in the distal direction from the proximal side of the second coupler 360.

The stoppers 354a, 354b of the first set and the stoppers 356a, 356b of the second set can be constructed of any suitable materials including polymers such as thermoplastics or thermosets, metals such as stainless steel, platinum, gold, nitinol, other alloys of metals, and any combination thereof.

With reference to FIG. 9, the first coupler 358 and the second coupler 360 are configured, e.g., sized, shaped, and/or constructed, to contact and apply an outwardly radial force to the tubular implant 320 in the collapsed state to grip the implant 320 in the lumen 304 of the catheter 302. The first coupler 358 and the second coupler 360 can be constructed from a material that is compressible and expandable. Alternatively, the first coupler 358 and/or the second coupler 3660 are constructed from an impressible material. By way of example, suitable materials for constructing the first coupler 358 and/or the second coupler 360 include polymeric materials e.g., elastomers such as silicone, thermosets, thermoplastics, thermoplastic urethanes, rubbers, or non-polymeric materials e.g., shape-memory metallic materials such as nitinol, stainless steel, cobalt chrome, etc.

The first coupler 358 and the second coupler 360 can be shaped and/or sized to provide a circumferential surface or surface segment conforming to the inner wall surface of the catheter 302 to allow the tubular implant 320 to be sandwiched or compressed between an inner surface of the catheter 302 and the first coupler 358 and the second coupler 360 respectively. By way of example, the first coupler 358 and/or the second coupler 360 may have a circular, semi-circular, oval, or other regular or irregular cross-sectional shape. In a specific embodiment of the disclosure, the first coupler 358 and/or the second coupler 360 are in the form of a friction pad made of an elastic polymeric material such as silicone having a circular cross-sectional shape.

According to some embodiments of the disclosure, the first coupler 358 and the second coupler 360 are configured to allow the delivery wire 352 e.g., the distal section 352b of the delivery wire 352 to pass therethrough. By way of example, the first coupler 358 and the second coupler 360 may be provided with a through-opening, channel, slot, or the like to allow the elongate delivery wire 352 to slide longitudinally in the lumen 304 of the catheter 302. In some embodiments, the through-opening, channel, slot, or the like allows the delivery wire 352 to rotate relative to the first and the second couplers 358, 360 in the lumen 304 of the catheter 302.

According to some embodiments of the disclosure, the first coupler 358 and/or the second coupler 360 may comprise a cylindrical tubular body e.g., in the form of a bushing. The cylindrical tubular body or bushing 358, 360 may have an outer surface configured for contacting the tubular implant 320 and a lumen allowing the delivery wire 352 to pass through. According to embodiments of the disclosure, the outer and inner diameters of the cylindrical bushing 358, 360 and the through-opening therein can be selected such that when the tubular implant 320 and the bushings 358, 360 are constrained in the lumen 304 of the catheter 302, the delivery wire 352 can still be torqued and rotated relative to the cylindrical bushing 358, 360. In some embodiments, the outer and inner diameters of the cylindrical bushing 358, 360 and the through-opening therein can be selected such that when the tubular implant 320 and the bushings are constrained in the lumen 304 of the catheter 302, the friction force between the compressed bushing 358, 360 and the delivery wire 352 is sufficient to prevent free rotation of the bushing 358, 360 relative to the delivery wire 352. After the first coupler 358 has exited the tip of the catheter 302, the delivery wire 352 can be torqued or rotated independently.

With reference to FIGS. 8-9, the delivery device 350 may include a protection cover 380. The protection cover 380 can protect the tubular implant 320, aid delivery and deployment of the tubular implant 320, and/or perform other functions. As discussed above, a braided stent may comprise a plurality of wires or filaments. The friction or abrasion of the wires or filaments against the inner wall of a catheter during delivery may cause wear, bending, breaking or other mechanical damage to the stent wires. The risk of damage to a braided stent increases when the stent is forced through a tortuous path of twists, turns, or bends. Damaged wires would compromise the integrity of the stent, hinder its opening or expansion, and prevent good apposition or contact between the expanded stent and the vessel wall. In addition, the ends of stent wires may penetrate into the inner liner of the catheter, digging out e.g., small polymeric particles and release them into the patient's body. For this and other reasons, a protection cover for a tubular implant would be highly desirable.

According to embodiments of the disclosure, the protection cover 380 can be made of a polymeric material such as polytetrafluoroethylene (PTFE), expanded polytetrafluoroethylene (ePTFE), polyurethane, polyethylene, or other biocompatible polymers. PTFE or ePTFE is known for its flexibility, biocompatibility, and smooth surface. Alternatively, the protection cover 380 may include a thin metallic mesh e.g., stainless steel or nitinol mesh for providing added mechanical strength and kink resistance.

With reference to FIGS. 8-9, the protection cover 380 may comprise a first end portion 382 and a second end portion 384. The first end portion 382 of the protection cover 380 may be fixedly coupled to the distal section 352b of the delivery wire 352, or fixedly coupled to a radiopaque marker 353 which is in turn affixed to the distal section 352b of the delivery wire 352. The second end portion 382 may be open to wrap or surround at least a portion of the end portion 321 of the tubular implant 320. The second end portion 384 of the protection cover 380 may be interposed between the end portion 321 of the implant 320 and the inner surface of the catheter 302. In some embodiments, the second end portion 384 of the protection cover 380 may radially wrap substantially the entire extremity end portion 321 of the tubular implant 320. Therefore, in some embodiments, the protection cover 380 may have a cone shape or configuration tapered from the second end portion 384 to the first end portion 382.

In some embodiments, the protection cover 380 may cover the end portion 321 of the stent 320 over a length ranging from 5-10 mm from the distal end extremity of the stent 320.

In some embodiments, the second end portion 384 of the protection cover 380 may be configured to provide a restrictive force that aids in maintaining the distal portion 322 of the stent 320 in a collapsed configuration. Alternatively, the protection cover 380 does not on its own provide a restraining force to maintain the stent 320 in a collapsed diameter.

In a specific embodiment, the protection cover 380 may comprise a malleable ePTFE layer having a first end portion 382 directly or indirectly affixed to the distal section 352 of the delivery wire 350 and a free second end portion 384 wrapping the end portion 321 of the stent 320 in the lumen 304 of the catheter 302.

In some embodiments, the protection cover 380 may comprise a continuous layer extending from the first end portion 382 to the second end portion 384. Alternatively, the protection cover 380 may include a slit sheath configuration or individual pieces end portions fixedly coupled to the delivery 352.

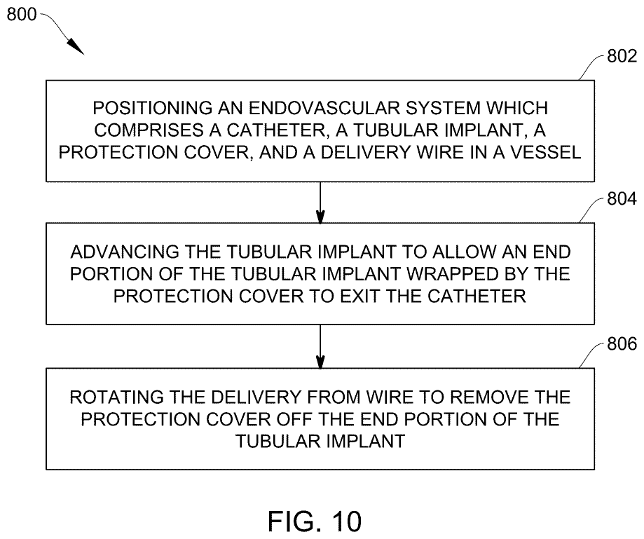

With reference now to FIGS. 10-12, a method 800 according to embodiments of the disclosure is now described. The method 800 can be used to deliver and deploy a tubular implant such as a braided stent or flow diverting stent in a cerebral vessel of a patient.

The method 800 may begin by positioning an endovascular system in a vessel of a patient, indicated at step 802 in FIG. 10. The endovascular system can comprise an embodiment described herein in conjunction with FIGS. 8-9 or any other suitable systems known in the art. In general, an example endovascular system 300 as shown in FIGS. 8-9 comprises a catheter 302 having a lumen 304, a tubular implant 320 constrained in the lumen 304 of the catheter 302, and a delivery device 350. The delivery device 350 comprises a delivery wire 352, one or more sets of stoppers 354a/354b, 356a/356b fixedly attached to the distal section 352b of the delivery wire 352, one or more couplers 358, 360 disposed between the one or more sets of stoppers 354a/354b, 356a/356b respectively, and a protection cover 380 comprising a first end portion 382 fixedly coupled to the delivery wire 352 and a second end portion 384 wrapping at least an end portion 321 of the tubular implant 320 in the lumen 304 of the catheter 302. In positioning the endovascular system 300, a guidewire and/or a guide catheter commonly known in the art may be used. Fluoroscopy known in the art may also be used in the positioning of the endovascular system 300. To obtain a desirable position of the endovascular system 300, the physician may advance and/or retract the tubular implant 320 several times.

The tubular implant 320 can be advanced or unsheathed by relative movement of the catheter 302 and the delivery wire 352. By way of example, the catheter 302 can be proximally withdrawn to expose the protection cover 380 and the distal end portion 321 of the implant 320. Alternatively, the delivery wire 352 can be pushed in a distal direction. The stoppers 354a, 354b of the first set and the stoppers 356a, 356b of the second set, which are affixed to the delivery wire 352, also move forward as the delivery wire 352 is pushed in the distal direction. The through-opening in the first and second couplers 358, 360 allows the delivery wire 352 to pass through. The first and second couplers 358, 360 can be configured, e.g., the inner and outer diameters of the couplers 358, 360, and the material making the couplers can be chosen such that the delivery wire 352 can longitudinally move and rotate relative to the first and second couplers 358, 360. According to embodiments of the disclosure, the first coupler 358 disposed between the first set of stoppers 354a, 354b and the second coupler 360 disposed between the second set of stoppers 356a, 356b are arranged such that when the delivery wire 352 is pushed in a distal direction to advance the stent 320, the proximal stopper 354b of the first set engages the first coupler 358. As such, a forward force is applied to the first coupler 358 by the proximal stopper 354b of the first set. The grip between the first coupler 358 and the braided stent 320 generates a pulling force, which pulls or stretches a portion 323 of the stent 320 proximal of the distal end portion 322 of the stent 320 e.g., the portion 323 between the first coupler 358 and the second coupler 360. As a result, the diameter of the stent portion 323 is reduced as the stent 320 is pulled or stretched. The contraction of the stent diameter reduces the normal force and/or surface area of the stent portion 323 against the inner surface of the catheter 302, thereby reducing the overall friction force between the stent 320 and the catheter 302. The slight elongation of the stent portion 323 also allows the proximal stopper 356b of the second set to engage the second coupler 360, allowing a forward force to be applied to the second coupler 360. Collectively, the forward force applied by the proximal stopper 354b of the first set, the pulling force in the distal direction generated by the first coupler 358 on the stent portion 323, and the forward force applied by the distal stopper 356b of the second set allow the stent 320 constrained in the lumen 304 of the catheter 302 to overcome the opposing friction and move forward. The pulling force in the distal direction generated by the first coupler 358 on the stent portion 323 reduces the overall resistance in advancing the stent 320 constrained in the lumen 304 of the catheter 302.

To retract the stent 320, the delivery wire 352 can be pulled in a proximal direction. The stoppers 354a, 354b of the first set and the stoppers 356a, 356b of the second set, which are affixed to the delivery wire 352, also move rearward as the delivery wire 352 is pulled in the proximal direction. According to embodiments of the disclosure, the first coupler 358 disposed between the first set of stoppers 354a, 354b and the second coupler 360 disposed between the second set of stoppers 356a, 356b are arranged such that when the delivery wire 352 is pulled in a proximal direction to retract the stent 320, the distal stopper 356a of the second set engages the second coupler 160. As such, a rearward force is applied to the second coupler 360 by the distal stopper 356a of the second set. The grip between the second coupler 360 and the stent 320 generates a pulling force, which pulls or stretches a portion 323 of the stent 320 distal of the proximal end portion 324 of the stent 320 e.g., the portion 323 between the first coupler 358 and the second coupler 360. As a result, the diameter of the stent portion 323 is reduced as the stent 320 is pulled or stretched. The contraction of the stent diameter reduces the normal force and/or surface area of the stent portion 323 against the inner surface of the catheter 302, thereby reducing the overall friction force between the stent 320 and the catheter 302. The slight elongation of the stent portion 323 also allows the distal stopper 354a of the first set to engage the first coupler 358, allowing a rearward force to be applied to the first coupler 358. Collectively, the rearward force applied by the distal stopper 356a of the second set, the pulling force in the proximal direction generated by the second coupler 360 on the stent portion 323, and the rearward force applied by the distal stopper 354a of the first set allow the stent 320 constrained in the lumen 304 of the catheter 302 to overcome the opposing friction and move rearward. The pulling force in the proximal direction generated by the second coupler 360 on the stent portion 323 reduces the overall resistance in retracting the stent 320 constrained in the lumen 304 of the catheter 302.

Therefore, according to embodiments of the disclosure the position of the proximal stopper 354b of the first set with respect to the first coupler 358 and the position of the proximal stopper 356b of the second set with respect to the second coupler 360 can be arranged to allow the proximal stopper 354b of the first set to engage the first coupler 358 before the proximal stopper 356b of the second set engages the second coupler 360 when the delivery wire 352 is pushed in a distal direction to advance an stent 320. As such, a pulling force in the distal direction can be generated by the first coupler 358 on a stent portion 323 between the first coupler 358 and the second coupler 360. The pulling force induces slight diametrical contraction of the stent portion 323, thereby reducing the overall static friction between the stent 320 and the catheter 302. The elongation of the stent portion 323 induced by the pulling force also allows the proximal stopper 356b of the second set to engage the second coupler 360, allowing a pushing force to be applied to the second coupler 360 to advance the stent 320.

Conversely, according to embodiments of the disclosure the position of the distal stopper 354a of the first set with respect to the first coupler 358 and the position of the distal stopper 356a of the second set with respect to the second coupler 360 can be arranged to allow the distal stopper 356a of the second set to engage the second coupler 360 before the distal stopper 354a of the first set engages the first coupler 358 when the delivery wire 352 is pulled in a proximal direction to retract an stent 320. As such, a pulling force in the proximal direction can be generated by the second coupler 360 on a stent portion 323 between the first coupler 358 and the second coupler 360. The pulling force induces slight diametrical contraction of the stent portion 323 between the first coupler 358 and the second coupler 360, thereby reducing the overall static friction between the stent 320 and the catheter 302. The elongation of the implant 320 induced by the pulling force also allows the distal stopper 354b of the first set to engage the first coupler 358, allowing a force to be applied to the first coupler 358 to retract the implant 320.

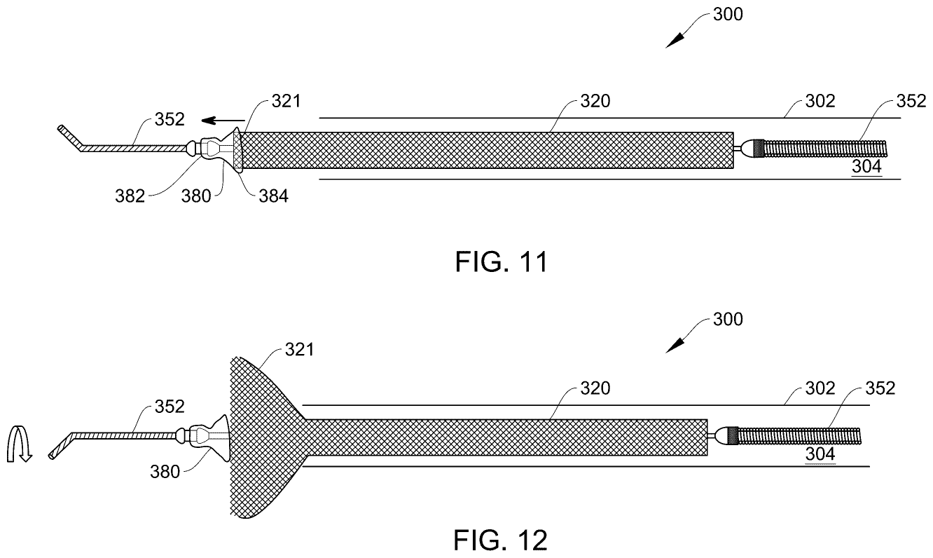

Returning to FIG. 10, according to embodiments of the method 800, the tubular implant 320 is advanced or unsheathed to allow an end portion of the tubular implant 320 wrapped by the protection cover 380 to exit the catheter 302, as indicated at step 804. For example, the delivery wire 352 can be advanced distally to allow the proximal stopper 354b to engage and apply a translating force to the coupler 358. The grip between the coupler 358 and the braided stent 320 allows the tubular implant 320 constrained in the lumen 304 of the catheter 302 to overcome the opposing friction and move forward. FIG. 11 schematically shows that at least the end portion 321 wrapped by the protection cover 380 exit of the lumen 304 of the catheter 302.

With reference to FIG. 10, according to embodiments of the method 800, the delivery wire 352 is rotated to remove the protection cover 380 off from the tubular implant 320, as indicated at step 806. By way of example, the physician can rotate the proximal section 352a of the delivery wire 352, which may be coupled to an operation handle. The torque applied at the proximal section 352a of the delivery wire 352 is transmitted to the distal section 352b of the delivery wire 352. Because the first end portion 382 of the protection cover 380 is fixedly coupled to the delivery wire 352, the rotation of the delivery wire 352 causes the protection cover 380 to rotate or wrap around the delivery wire 352, pulling the second end portion 384 of the protection cover 380 off from the end portion 321 of the tubular implant 320, thereby releasing the end portion 321 of the implant 320 from the constraint of the protection cover 320. FIG. 12 schematically shows that after the constraint of the protection cover 320 is removed, the implant portion 321 exiting the catheter 302 expands to an expanded configuration.

Various embodiments of an endovascular system, a delivery device, and a method have been described with reference to figures. It should be noted that an aspect described in conjunction with a particular embodiment is not necessarily limited to that embodiment and can be practiced in any other embodiments. The figures are intended for illustration of embodiments but not for exhaustive description or limitation on the scope of the disclosure. Alternative structures, components, and materials will be readily recognized as being viable without departing from the principle of the claimed invention.

All technical and scientific terms used herein have the meaning as commonly understood by one of ordinary skill in the art unless specifically defined otherwise. As used in the description and appended claims, the singular forms of “a,” “an,” and “the” include plural references unless the context clearly dictates otherwise. The term “or” refers to a nonexclusive “or” unless the context clearly dictates otherwise. The term “proximal” and its grammatical equivalents refer to a position, direction or orientation towards the user or physician's side. The term “distal” and its grammatical equivalents refer to a position, direction, or orientation away from the user or physician's side. The designations “rearward,” “forward,” and the like are not meant to limit the referenced component to a specific orientation. It will be appreciated that such designations refer to the orientation of the referenced component as illustrated in the Figures; the systems and devices of the disclosure can be used in any orientation suitable to the user. The term “first” or “second” etc. may be used to distinguish one element from another in describing various similar elements. It should be noted the terms “first” and “second” as used herein include references to two or more than two. Further, the use of the term “first” or “second” should not be construed as in any particular order unless the context clearly dictates otherwise. The order in which the method steps are performed may be changed in alternative embodiments. One or more method steps may be skipped altogether, and one or more optional steps may be included. All numeric values are provided for illustration and assumed to be modified by the term “about,” whether explicitly indicated or not. The term “about” generally refers to a range of numbers that one of skill in the art would consider equivalent to the recited value e.g., having the same function or result. The term “about” may include numbers that are rounded to the nearest significant figure. The recitation of a numerical range by endpoints includes all numbers within that range.

Those skilled in the art will appreciate that various other modifications may be made. All these or other variations and modifications are contemplated by the inventors and within the scope of the invention.

What is claimed is:

1. An endovascular system, comprising:
a catheter having a lumen;
a tubular implant having a collapsed state for being disposed in the lumen of the catheter and an expanded state when unconstrained by the catheter; and
a delivery device operable to deliver the tubular implant through the catheter, wherein the delivery device comprises:
a delivery wire having a distal section and a proximal section;
a set of stoppers comprising a distal stopper and a proximal stopper fixedly attached to the distal section of the delivery wire respectively;
a coupler disposed between the distal stopper and the proximal stopper and configured to apply an outwardly radial force to a distal portion of the tubular implant in the lumen of the catheter to grip the tubular implant, the coupler comprising a through-opening allowing the distal section of the delivery wire to pass through; and
a protection cover comprising a first end portion fixedly coupled to the distal section of the delivery wire and a second end portion wrapping at least an end portion of the tubular implant in the lumen of the catheter,
wherein the coupler is configured to allow the delivery wire to slide longitudinally relative to the coupler in the lumen of the catheter to thereby allow the proximal stopper and/or the distal stopper to engage and apply a translating force to the coupler to advance and/or withdraw the tubular implant, and to allow the delivery wire to rotate relative to the coupler to thereby remove the protection cover off from the tubular implant.

2. The endovascular system of claim 1, wherein the distal section of the delivery wire comprises a tip segment angled away from a longitudinal axis of the catheter, whereby the rotation of the delivery wire allows the tip segment to change orientation to facilitate delivery and/or deployment of the tubular implant in a tortuous vessel of a patient and/or avoid entrance to a branch off a vessel the tubular implant is being deployed in.

3. The endovascular system of claim 1, wherein the protection cover is constructed of a polymeric material comprising expanded polytetrafluoroethylene (ePTFE).

4. The endovascular system of claim 1, wherein the coupler is constructed of an elastic polymeric material.

5. The endovascular system of claim 1, wherein the coupler is in the form of a cylindrical tubular body constructed from silicone.

6. The endovascular system of claim 1, wherein the tubular implant comprises a braided structure.

7. The endovascular system of claim 6, wherein the braided structure is constructed of two or more composite wires each comprising an inner core of a first material and an outer sheath of a second material different from the first material.

8. The endovascular system of claim 7, wherein the first material of the inner core comprises a nickel-cobalt alloy, and the second material of the outer sheath comprises a nickel-titanium alloy.

9. The endovascular system of claim 8, wherein the two or more composite wires comprise a fill percentage of the inner core ranging from about 5 to 30 percent.

10. The endovascular system of claim 1, wherein the set of stoppers comprises a first set of the stoppers and a second set of stoppers, the coupler comprises a first coupler disposed between the first set of the stoppers and a second coupler disposed between the second set of the stoppers, wherein the first coupler is configured to apply an outwardly radial force to the distal portion of the tubular implant in the lumen of the catheter, and the second coupler is configured to apply an outwardly radial force to a proximal portion of the tubular implant in the lumen of the catheter.

11. The endovascular system of claim 1, further comprising a bumper fixedly coupled to the delivery wire and located adjacent to a proximal end of the tubular implant.

12. A method of delivering a tubular implant, comprising:
positioning an endovascular system in a vessel of a patient, the endovascular system comprising a catheter having a lumen, a tubular implant constrained in the lumen of the catheter, and a delivery device, wherein the delivery device comprises a delivery wire, a set of stoppers comprising a distal stopper and a proximal stopper fixedly attached to the delivery wire, a coupler between the distal stopper and the proximal stopper configured to contact and apply an outwardly radial force to a distal portion of the tubular implant in the lumen of the catheter to grip the tubular implant, and a protection cover comprising a first end portion fixedly coupled to the delivery wire and a second end portion wrapping at least an end portion of the tubular implant in the lumen of the catheter;
advancing or unsheathing the tubular implant by relative movement of the catheter and the deliver wire to allow the proximal stopper to engage and apply a translating force to the coupler, whereby the end portion of the tubular implant wrapped by the second end portion of the protection cover exits the lumen of the catheter; and
rotating the delivery wire to allow the protection cover to twist, whereby the second end portion of the protection cover is removed off from the end portion of the tubular implant.

13. The method of claim 12, wherein the protection cover is constructed of a flexible polymeric material.

14. The method of claim 12, wherein the protection cover is constructed of a polymeric material comprising expanded polytetrafluoroethylene (ePTFE).

15. The method of claim 12, wherein the tubular implant comprises a braided structure.

16. The method of claim 15, wherein the braided structure is constructed of two or more composite wires each comprising an inner core of a first material and an outer sheath of a second material different from the first material.

17. The method of claim 16, wherein the first material of the inner core comprises a nickel-cobalt alloy, and the second material of the outer sheath comprises a nickel-titanium alloy.

18. The method of claim 17, wherein the two or more composite wires comprise a fill percentage of the inner core ranging from about 5 to 30 percent.

19. The method of claim 12,
wherein the set of stoppers comprises a first set of the stoppers and a second set of stoppers, the coupler comprises a first coupler disposed between the first set of the stoppers and a second coupler disposed between the second set of the stoppers, wherein the first coupler is configured to apply an outwardly radial force to the distal portion of the tubular implant in the lumen of the catheter, and the second coupler is configured to apply an outwardly radial force to a proximal portion of the tubular implant in the lumen of the catheter; and
wherein in the relative movement of the catheter and the delivery wire, a proximal stopper of the first set engages and applies the translating force to the first coupler, and a proximal stopper of the second set engages and applies a translating force to the second coupler.

20. The method of claim 19, wherein in the relative movement of the catheter and the delivery wire, the proximal stopper of the first set engages the first coupler before the proximal stopper of the second set engages the second coupler.
